(19) United States
(12) Patent Application Publication
(10) Pub. No.: US 2024/0182948 A1
(43) Pub. Date: Jun. 6, 2024

(54) METHODS AND COMPOSITIONS FOR DETECTION OF TUMOR DNA

(71) Applicants: The Broad Institute, Inc., Cambridge, MA (US); Massachusetts Institute of Technology, Cambridge, MA (US); The General Hospital Corporation, Boston, MA (US)

(72) Inventors: Viktor A. Adalsteinsson, Cambridge, MA (US); Nir Hacohen, Cambridge, MA (US); Sangeeta N. Bhatia, Cambridge, MA (US); Sahil Patel, Cambridge, MA (US); Zhenyi An, Cambridge, MA (US)

(73) Assignees: The Broad Institute, Inc., Cambridge, MA (US); Massachusetts Institute of Technology, Cambridge, MA (US); The General Hospital Corporation, Boston, MA (US)

(21) Appl. No.: 18/285,103

(22) PCT Filed: Mar. 30, 2022
(86) PCT No.: PCT/US2022/022682

§ 371 Date: Sep. 29, 2023

Related U.S. Application Data
(60) Provisional application No. 63/168,244, filed on Mar. 30, 2021.

(51) Int. Cl. C12Q 1/6806; A61K 31/4706; A61K 31/713; C12Q 1/6886

(52) U.S. Cl. CPC C12Q 1/6806; A61K 31/4706; A61K 31/713; C12Q 1/6886

(57) ABSTRACT

This disclosure provides a method and compositions for substantially increasing the concentration of DNA in macrophages of a patient. By administering to a patient one or more agents which prevent the activity of deoxyribonucleases within lysosomes of macrophages, degradation of DNA phagocytosed by macrophages is temporarily blocked, permitting its accumulation. This strategy has the potential to enhance the detection of genetic biomarkers from cells typically phagocytosed by macrophages, such as tumor cells, and thus has applications for early or residual detection of cancer.

### RELATED APPLICATIONS AND INCORPORATION BY REFERENCE

This International PCT Application claims priority to U.S. Provisional Application No. 63/168,244, filed Mar. 30, 2021, the contents of which are incorporated by reference.

### BACKGROUND

### Field of the Disclosure

The present disclosure relates to improved tumor detection methods, compositions, and kits. The disclosure further relates to methods, compositions, and kits for enhancing the amount of tumor-derived DNA (or simply “tumor DNA”) that may be obtained from a biological source, thereby improving cancer detection. Still further, the disclosure relates to methods, compositions, and kits for enhancing the amount of tumor-derived DNA obtainable from a biological sample, including where the tumor-derived DNA is from an early-stage cancer, e.g., early-stage lung cancer, and is present in low abundance, thereby improving cancer detection of early-stage cancers.

### Background of the Disclosure

Cancer is often a fatal disease. Modern medicine has developed many modes of treating cancer, including surgical removal of tumors, chemotherapy, and immunological therapy. However, the key to effective treatment is often dependent upon early detection. Lung cancer is no exception.

Indeed, lung cancer is the leading cause of cancer-related mortality, with greater than 1.7 million annual deaths worldwide. Survival is strongly associated with the stage of cancer that exists at the time of diagnosis. In particular, the five-year overall survival rate of lung cancer is 56% in localized cancer, 29% in regional cancer, and 5% in distant-stage lung cancer disease. Clearly, the five-year survival rating is significantly higher for early-stage cancer (e.g., stage I cancer). Unfortunately, fewer than 20% of early-stage lung cancers are even diagnosed^{1,2}.

A number of diagnostic tests are available for determining the presence of cancer, some of which can be used in the detection of lung cancer. These tests (and which can depend upon the location of the tumor) include surgical biopsies, computed axial tomography (CAT or CT scans), bronchoscopy, magnetic resonance imaging (MRI) scans, ultrasound scans, positron emission tomography (PET) scans, bone marrow testing, barium swallow tests, endoscopy, cystoscopy, T/Tn antigen tests, mammography, and other tests. Each of these tests has advantages and disadvantages. For example, lung cancer screening via low-dose computed tomography (CT) has proven to reduce lung cancer mortality in high-risk patients. However, many patients who are ultimately diagnosed with lung cancer would not fit current chest CT screening criteria.

Furthermore, there is an increasing number of incidental nodules (i.e., small growths in a tissue, e.g., pulmonary nodules found in the lung, that can be cancerous or non-cancerous) being identified during imaging studies obtained for alternative indications. This presents major diagnostic dilemmas, including how to better identify patients at risk for lung cancer, and what to do about pulmonary nodules once they are discovered. Currently, the only ways to tell which pulmonary nodules are cancerous is to “watch and wait” or perform an invasive procedure. Yet, each harbors risk for delayed diagnosis or unnecessary harm, while it remains exceedingly difficult to accurately biopsy peripheral nodules—where 70% of lung cancers develop. With more than 2.1 million new lung cancer cases worldwide each year, better lung cancer diagnostics are urgently needed, particularly of early-stage lung cancers.

Since lung cancer patients do not often have detectable symptoms until later stage disease, diagnosis of early-stage disease presents many challenges. Indeed, many of the methods of cancer detection mentioned above, particularly ultrasound or CAT scans and bronchoscopy, are not effective in detecting early-stage lung cancers. Many physicians have tried sputum cytology and CT scans, coupled with bronchoscopy for early cancer detection, as well as biochemical approaches to identify and quantify various lung cancer biomarkers from blood serum (e.g., using liquid biopsies) based on detection of cell-free tumor DNA. In this approach, however, the sensitivity is still low because cell-free tumor DNA is often presents in the blood or blood plasma at low concentrations. Moreover, low concentrations of cell-free tumor DNA in the blood is particularly true for earlier-stage cancers.

Therefore, new approaches that result in increased sensitivity in diagnosing cancer, including in particular, diagnosing early-stage cancers, such as early-stage lung cancer, are needed in the art.

### SUMMARY OF THE INVENTION

The present disclosure relates in part to the surprising finding that phagocytic leukocytes, e.g., tumor-associated macrophages, involved in the homeostatic clearance of apoptotic cells in a developing tumor, e.g., a lung tumor, may be leveraged as a vehicle or reservoir for the accumulation of large amounts of tumor-derived DNA (i.e., DNA whose origin is a tumor cell) to facilitate the analysis of same in the diagnosis cancer, and in particular cancers that are early-stage and have otherwise low levels of cell-free tumor DNA (i.e., a type of tumor-derived DNA) circulating in the blood.

In one embodiment, the methods, compositions, and kits described herein, involve collecting or otherwise obtaining phagocytic leukocytes, e.g., tumor-associated macrophages, involved in the homeostatic clearance of apoptotic tumor cells and isolating from the cells cytoplasmic DNA, which includes the macrophage-ingested tumor-derived DNA. In some embodiments, the tumor-associated macrophages may be obtained from the tumor microenvironment of a tumor, e.g., a fluid specimen that is a part of or is in contact with a tumor microenvironment. As used herein, the “tumor microenvironment” refers to the normal cells, molecules, fluid, and blood vessels that surround and feed a tumor. In one embodiment, it is therefore proposed that sampling biologic fluid specimens in contact with the tumor microenvironment and collecting the associated tissue-resident macrophages could allow one to acquire much more tumor-derived DNA than would be possible with other methods, such as liquid biopsies from blood samples. One of ordinary skill in the art will appreciate that a tumor is not simply a group of cancer cells, but rather a heterogeneous collection of infiltrating and resident host cells, secreted factors, and extracellular matrix. Tumor cells stimulate significant molecular, cellular, and physical changes within their host tissues to support tumor growth and progression. Further, a tumor microenvironment is a complex and continuously evolving entity. Still further, the composition of the tumor microenvironment varies between tumor types, but hallmark features include, for example, immune cells (including tumor-associated macrophages), stromal cells, blood vessels, and extracellular matrix. It is believed that the tumor microenvironment is not just a silent bystander, but rather an active promoter of cancer progression. Early in tumor growth, a dynamic and reciprocal relationship develops between cancer cells and components of the tumor microenvironment that supports cancer cell survival, local invasion and metastatic dissemination. Also, to overcome a hypoxic and acidic microenvironment, the tumor microenvironment coordinates a program that promotes angiogenesis to restore oxygen and nutrient supply and remove metabolic waste. Tumors become infiltrated with diverse adaptive and innate immune cells that can perform both pro- and anti-tumorigenic functions.

In yet another embodiment, the methods, compositions, and kits described herein, involve collecting or otherwise obtaining phagocytic leukocytes, e.g., tumor-associated macrophages, involved in the homeostatic clearance of apoptotic tumor cells and isolating from the cells a cytoplasmic fraction that comprises lysosomes, and then isolating the tumor-derived DNA from the lysosomes.

In another embodiment, the methods, compositions, and kits described herein can first involve administering a subject with an effective amount of one or more agents which inhibit, reduce, or otherwise minimize the degradation of tumor-derived DNA (released from said apoptotic tumor cells) by the phagocytic leukocytes, e.g., tumor-associated macrophages. In various embodiments, the one or more agents comprises a nuclease inhibitor, which inhibits the activity of a nuclease (e.g., a DNase II) associated with the phagocytic leukocytes, e.g., tumor-associated macrophages, which are involved in nuclease-dependent digestion of the tumor-derived DNA. In various other embodiments, the one or more agents comprises a pH-adjusting agent, which increases or lowers the pH of the microenvironment of the phagocytic leukocytes (or of their lysosomes therein) such that the nucleases associated therewith (e.g., DNase II) are inhibited from degrading the tumor-derived DNA.

In various embodiments, the methods, compositions, and kits described herein, involve contacting the phagocytic leukocytes, e.g., tumor-associated macrophages, involved in the homeostatic clearance of apoptotic tumor cells, with an effective amount of one or more agents which inhibit, reduce, or otherwise minimize the degradation of tumor DNA (released from said apoptotic tumor cells) by the phagocytic leukocytes, e.g., tumor-associated macrophages. In various embodiments, the one or more agents comprises a nuclease inhibitor, which inhibits the activity of a nuclease (e.g., a DNase II) associated with the phagocytic leukocytes, e.g., tumor-associated macrophages, which are involved in nuclease-dependent digestion of the tumor-derived DNA. In various other embodiments, the one or more agents comprises a pH-adjusting agent, which increases or lowers the pH of the microenvironment of the phagocytic leukocytes (or of their lysosomes therein) such that the nucleases associated therewith (e.g., DNase II) are inhibited from degrading the tumor-derived DNA.

In one aspect, the disclosure provides a method for enhancing tumor detection in a tissue, comprising administering an agent that increases the accumulation of tumor-derived DNA in a tumor-associated macrophage (or in the lysosome thereof) or population thereof, and evaluating the tumor-derived DNA, thereby detecting the tumor. In another aspect, the disclosure provides a method for increasing the recovery of tumor-derived DNA from a tumor-associated macrophage (or from lysosomes thereof), comprising administering an agent that increases the accumulation of tumor-derived DNA in a macrophage (or in lysosomes thereof) and recovering the tumor-derived DNA from said tumor-associated macrophage (or from the lysosomes of said macrophage). In still another aspect, the disclosure provides pharmaceutical compositions for increasing tumor-derived DNA in a population of tumor-associated macrophages (or in the lysosome fraction) comprising an agent and a pharmacologically acceptable excipient, wherein the agent results in the increased accumulation of tumor-derived DNA in a tumor-associated macrophage (or in the lysosomal fraction) as compared to in the absence of the agent. In yet another aspect, the disclosure provides a kit for the detection of phagocytosed DNA from a population of tumor-associated macrophages, comprising an agent for increasing phagocytosed DNA in a population of tumor-associated macrophages (or in their lysosomes), and instructions for use. In yet another aspect, the disclosure provides a method for detecting a tumor in a tissue, comprising collecting a macrophage or population thereof from the tissue, and evaluating the accumulated tumor-derived DNA from the macrophage (or from the lysosome fraction), thereby detecting the tumor.

In various embodiments, the one may extract the tumor-derived DNA from the recovered tumor-associated macrophages. In other embodiments, one may extract the lysosomes or a cytoplasmic fraction comprising the lysosomes from the recovered tumor-associated macrophages, and then extract the tumor-derived DNA from the lysosomes or the fraction comprising the lysosomes.

Without being bound by theory, a significant number of cancer cells undergo apoptosis in a developing tumor, and release tumor-derived DNA comprising distinctive DNA signatures that may be targeted by one or more detection agents (e.g., a detection antibody with a detectable moiety, such as a fluorescence or enzymatic tag) to detect the tumor and/or its properties (e.g., the cancer stage). However, only a tiny fraction of tumor-derived DNA spills into the bloodstream in the form of “cell-free” tumor-derived DNA. The vast majority of tumor-derived DNA is cleared via efferocytosis of apoptotic cancer cells or apoptotic bodies by phagocytic leukocytes (e.g., macrophages associated with a tumor), especially in parts of the body where developing tumors and macrophages are in close proximity, such as in the airway lumen where lung tumors develop and interact with alveolar macrophages. Apoptotic cells degrade their genomes into nucleosomal fragments via enzymes, such as caspase-activated DNase and present signals for engulfment by phagocytes, especially tumor-associated macrophages. However, this nucleosomal DNA does not become fully digested until it enters phagocytic lysosomes.

The inventors have recognized that this presents an opportunity to recover tumor-derived DNA from phagocytic lysosomes of such phagocytic leukocytes (e.g., tumor-associated macrophages) if the activity of deoxyribonucleases within these lysosomes and/or macrophages is inhibited. The inventors have found that this approach enables significantly more tumor-derived DNA to be recovered from a subject for analysis than would otherwise be possible and would thus have considerable applications for more accurate cancer detection. In certain embodiments, however, the inventors have found that tumor-derived DNA may be obtained from the lysosomes of such tumor-associated macrophages without inhibiting the activity of deoxyribonucleases (e.g., DNaseII) in the macrophages.

In one aspect, the disclosure provides a method for increasing the concentration of tumor-derived DNA in a macrophage or population thereof, comprising administering to a subject an effective amount of an agent that results in an increase in the accumulation of tumor-derived DNA in the macrophage or population thereof in the subject.

In another aspect, the disclosure provides a method for increasing the recovery of tumor-derived DNA from a macrophage or population thereof, comprising: administering to a subject an effective amount of an agent that increases the accumulation of tumor-derived DNA in a macrophage or population thereof in a subject; and obtaining the tumor-derived DNA from the macrophage or population thereof.

In still another aspect, the disclosure provides a method for enhancing tumor detection in a tissue, comprising administering an agent that increases the accumulation of tumor-derived DNA in a macrophage or population thereof in the tissue, and evaluating the accumulated tumor-derived DNA, thereby detecting the tumor.

In yet another aspect, the disclosure provides a pharmaceutical composition for increasing the concentration of tumor-derived DNA, comprising: an agent capable of increasing the concentration of tumor-derived DNA in a macrophage or population thereof; and a pharmacologically acceptable excipient.

In still another aspect, the disclosure provides a kit for increasing the concentration of phagocytosed DNA in a population of macrophages in a subject, comprising: a pharmaceutical composition comprising an agent capable of increasing the concentration of tumor-derived DNA in the macrophage or population thereof; optionally a device for administering said composition; and instructions for administering the composition.

In one embodiment, the concentration of tumor-derived DNA in the macrophage or population thereof is increased by up to 2-fold, up to 3-fold, up to 4-fold, up to 5-fold, up to 10-fold, up to 25-fold, up to 50-fold, up to 100-fold, up to 200-fold, up to 300-fold, up to 400-fold, up to 500-fold, up to 600-fold, up to 700-fold, up to 800-fold, up to 900-fold, or up to 1000-fold.

In another embodiment, the agent results in a reduction in the rate of tumor-derived DNA degradation in the macrophage or population thereof. The rate of tumor-derived DNA degradation in the macrophage or population thereof can be reduced by up to 5%, up to 10%, up to 15%, up to 20%, up to 25%, up to 30%, up to 35%, up to 40%, up to 45%, up to 50%, up to 55%, up to 60%, up to 65%, up to 70%, up to 75%, up to 80%, up to 85%, up to 90%, up to 95%, up to 99%, or up to 100%, as compared to the rate of tumor-derived DNA degradation in a macrophage or population thereof in the absence of the agent.

The agent can be a small molecule that reduces the expression and/or activity of one or more deoxyribonucleases in the macrophage or population thereof. For example, the agent can be a DNase II inhibitor.

In other embodiments, the agent is a small molecule that results in an increase in the lysosomal pH of the macrophage or population thereof. For example, the agent can be chloroquine or a derivative thereof.

The agent can also be a nucleic acid that reduces the expression and/or activity of one or more deoxyribonucleases in the macrophage or population thereof. For example, the agent can be an siRNA or shRNA that selectively binds to a mRNA transcript encoding DNase II.

In various embodiments, the agent is administered intravenously, orally, or through inhalation.

In various embodiments, the increased concentration of tumor-derived DNA in the macrophage or population thereof is due to an increased concentration of tumor-derived DNA in the lysosomes of the macrophage or population thereof.

In certain embodiments, the subject is a human patient who has or is suspected of having, or is at risk for a disease. The disease can be cancer, and in particular, early-stage cancer. In a particular embodiment, the cancer is an early-stage lung cancer. In another particular embodiment, the cancer is an early-stage lung cancer with one or more peripheral nodes.

The cancer may also be selected from: colorectal cancer, lung cancer, breast cancer, pancreatic cancer, prostate cancer, bladder cancer, kidney cancer, thyroid cancer, uterine cancer, cervical cancer, ovarian cancer, testicular cancer, esophageal cancer, stomach cancer, liver cancer, brain cancer, peritoneal cancer, lymphoma, leukemia, multiple myeloma, neuroblastoma, osteosarcoma, and soft tissue sarcoma.

The method herein may also involve collecting macrophages, and then isolating the tumor-derived DNA from the macrophages. In further embodiments, the lysomes from the macrophages many be collected. DNA may be isolated from the lysosomes, including tumor-derived DNA accumulated in the lysosomes.

In various embodiments, once the tumor-derived DNA is obtained, the tumor-derived DNA may be assayed, detected, or otherwise evaluated by one or more methods of techniques for detecting tumor-derived DNA, or a biomarker present therein.

In various embodiments, the agent is administered by inhalation as a formulation comprising aerosolized microspheres comprising said agent.

In still another aspect, the disclosure provides a method for detecting a tumor in a tissue, comprising collecting a macrophage or population thereof from the tissue, and evaluating the accumulated tumor-derived DNA, thereby detecting the tumor. In some embodiments, the macrophage or population thereof is collected from one or more biological samples, such as a sample comprising sputum. In some embodiments, the macrophage or population thereof are collected by sputum induction or bronchoalveolar lavage (BAL)

In some embodiments, the tissue is a tissue occurring in a subject. In certain embodiments, the subject is a human.

In further embodiments, the method further comprising administering to the subject an effective amount of an agent that results in an increase in the accumulation tumor-derived DNA, prior to the collection of the macrophage or population thereof from the tissue.

In one embodiment, the agent results in an increase in the concentration of tumor-derived DNA in the macrophage or population thereof. The concentration of tumor-derived DNA in the macrophage or population thereof can be increased by up to 2-fold, up to 3-fold, up to 4-fold, up to 5-fold, up to 10-fold, up to 25-fold, up to 50-fold, up to 100-fold, up to 200-fold, up to 300-fold, up to 400-fold, up to 500-fold, up to 600-fold, up to 700-fold, up to 800-fold, up to 900-fold, or up to 1000-fold, as compared to the concentration of tumor-derived DNA in a macrophage or population thereof in the absence of the agent.

In another embodiment, the agent results in a reduction in the rate of tumor-derived DNA degradation in the macrophage or population thereof. The rate of tumor-derived DNA degradation in the macrophage or population thereof can be reduced by up to 5%, up to 10%, up to 15%, up to 20%, up to 25%, up to 30%, up to 35%, up to 40%, up to 45%, up to 50%, up to 55%, up to 60%, up to 65%, up to 70%, up to 75%, up to 80%, up to 85%, up to 90%, up to 95%, up to 99%, or up to 100%, as compared to the rate of tumor-derived DNA degradation in a macrophage or population thereof in the absence of the agent.

The agent can be a small molecule that reduces the expression and/or activity of one or more deoxyribonucleases in the macrophage or population thereof. For example, the agent can be a DNase II inhibitor.

In other embodiments, the agent is a small molecule that results in an increase in the lysosomal pH of the macrophage or population thereof. For example, the agent can be chloroquine or a derivative thereof.

The agent can also be a nucleic acid that reduces the expression and/or activity of one or more deoxyribonucleases in the macrophage or population thereof. For example, the agent can be an siRNA or shRNA that selectively binds to a mRNA transcript encoding DNase II.

In various embodiments, the agent is administered intravenously, orally, or through inhalation. In certain embodiments, the agent is administered via a formulation comprising aerosolized microspheres comprising said agent. In some embodiments, the macrophage or population thereof is collected at least 1 hour after administering the agent.

In various embodiments, the method further comprises attenuating macrophage adherence prior to collection of the macrophage or population thereof, thereby increasing the yield of said macrophage or population thereof. In various embodiments, the method further comprises increasing efferocytosis of tumor-derived DNA by the macrophage or population thereof, prior to collection of the macrophage or population thereof.

In certain embodiments, the tissue is lung tissue. In certain embodiments, the macrophage or population thereof is an alveolar macrophage or population thereof. In certain embodiments, the tumor is a lung cancer tumor.

In some embodiments, the method further comprises isolating the tumor-derived DNA from the macrophage or population thereof, prior to evaluation of the tumor-derived DNA. In some embodiments, the method further comprises isolating the lysosomes from the macrophage or population thereof. In some embodiments, the method further comprises isolating the tumor-derived DNA from the lysosomes.

### BRIEF DESCRIPTION OF DRAWINGS

The following drawings form part of the present specification and are included to further demonstrate certain aspects of the present disclosure, which can be better understood by reference to one or more of these drawings in combination with the detailed description of specific embodiments presented herein.

FIG. 1 presents a schematic illustrating methods for assessing phagocytosis of tumor-derived DNA by macrophages. The upper panel shows an experimental design for assessing tumor-derived DNA phagocytosis and subsequent recovery from Wild Type and Dnase2a−/− Bone Marrow-Derived Macrophages (BMDM). The lower panel shows an experimental design for assessing tumor-derived DNA phagocytosis and recovery in an in vivo model. C57B16/J mice are inoculated with K-rasLSL-G12D/+; p53fl/fl Luciferase/GFP expressing (KPLG) lung adenocarcinoma cells via tail vein injection. At medium to high tumor burden, blood and bronchoalveolar lavage fluid (BALF) samples are obtained. BALF samples are separated into three components: CD45+ myeloid cells, CD45− non-myeloid cells, and BALF supernatant. Cellular components are then fractionated, and samples are used for DNA quantification and sequencing.

FIGS. 2A and 2B: Macrophages engulf and digest DNA from tumor cells. FIG. 2A: Schematic illustrating an experimental procedure for testing phagocytosis of tumor cell DNA by Bone Marrow-Derived Macrophages (BMDMs). FIG. 2B: Apoptotic murine lung adenocarcinoma KPLG cells were labeled with Hoechst 33342 and co-incubated with BMDMs at a 1:1 ratio. Live-cell imaging was performed after 30 minutes of co-incubation.

FIG. 3 presents a schematic illustrating how tumor cells in the lung are typically phagocytosed by lung alveolar macrophages (LAM). Upon treatment with an agent that disrupts lysosomal digestion of tumor cells and the genetic content they contain (e.g., by treatment with an inhaled DNase II inhibitor), LAM accumulate tumor-derived DNA and may be collected through sputum induction or bronchoalveolar lavage. Collected DNA within LAM may then be sequenced and analyzed, such as for tumor fraction or copy number alterations of genes and/or chromosomes.

FIGS. 4A-4E: Examples of techniques for highly sensitive sequencing of cell-free cancer DNA. FIG. 4A: Ultra-low pass whole genome sequencing can detect copy number alterations from even minute amounts of cancer DNA. FIG. 4B: Copy number alterations determined using trace amounts of cancer DNA correlate closely with copy number alterations determined using >10× the amount of cancer DNA for ultra-low pass whole genome sequencing. FIG. 4C: Tumor fraction may be accurately estimated even by sequencing trace amounts of cancer DNA. FIG. 4D: Ultra-low pass whole genome sequencing of cell free cancer DNA accurately determines the genome of tumor cells. FIG. 4E: Whole-exome sequencing of cell free cancer DNA can be used to determine the majority of mutations found in surgical tumor biopsies.

FIGS. 5A-5C: Examples of detection of cancer cell DNA from in vitro cell culture models. FIG. 5A: Cancer DNA obtained from the culture media of cancer cells exhibits similar fragmentation patterns and histone occupancy as DNA isolated from the cells themselves. FIG. 5B: Cancer cells release relatively low levels of genomic DNA into the extracellular environment, compared to that which can be obtained from the cells themselves. FIG. 5C: Individual cancer cells may be isolated and sequenced using single-cell sequencing methods.

FIGS. 6A and 6B: Development of DNase II knockout (KO) macrophages. FIG. 6A: Sequence information for guide RNAs (gRNAs) used with CRISPR targeting DNase II in BMDMs. FIG. 6B: Evaluation of mRNA levels of DNase II (DNase 2a), Ifit3, and Isg15 in wild-type control and DNase II KO BMDMs.

FIGS. 7A and 7B: Digestion of tumor-derived DNA by macrophages is mediated by DNase II. FIG. 7A: Fluorescent imaging showing that knockout of DNase II in bone marrow-derived macrophages (BMDMs) results in an increase in undigested KPLG DNA within macrophages. FIG. 7B: Quantification of fluorescent imaging shown in FIG. 7A.

FIGS. 8A-8C: Recovery of tumor-derived DNA from the cytoplasmic fraction of WT and DNase II KO macrophages. FIG. 8A: DNA mass recovered from the cytoplasmic fraction of different quantities of BMDMs co-incubated with apoptotic KPLG cells. FIG. 8B: The total DNA mass recovered from each fractionated cellular compartments in wild-type (WT) control or DNase II KO BMDMs. N: nuclear; M: mitochondrial; C: cytoplasmic. FIG. 8C: Primer sequences for KRAS WT and KRAS G12D detection by TaqMan quantitative PCT (qPCR). FIG. 8D: The calculated ratio of KRAS G12D to KRAS WT in control WT BMDMs and DNase II KO BMDMs in cytoplasmic fractions. FIG. 8E: The calculated ratio of KRAS G12D to KRAS WT in wild-type or DNase II KO macrophages, across all cellular fractions and time points tested. N: nuclear; M: mitochondrial; C: cytoplasmic; 0: time point 0; 2: 2 hours after washing away apoptotic KPLG cells.

FIGS. 9A-9D: Chloroquine (CQ) treatment inhibits digestion of cytoplasmic tumor-derived DNA by macrophages. FIG. 9A: Cytoplasmic DNA mass recovered from macrophages with or without 25 μM CQ treatment for 3 hours. FIG. 9B: Nuclear DNA mass recovered from macrophages with or without 25 μM CQ treatment for 3 hours. FIG. 9C: Cytoplasmic KRAS G12D to WT ratio. Apoptotic KPLG cells were washed away after 1 hour of co-incubation with macrophages before CQ treatment. FIG. 9D: Nuclear KRAS G12D to WT ratio. Apoptotic KPLG cells were washed away after 1 hour of co-incubation with macrophages before CQ treatment.

FIGS. 10A-10C: In vivo studies of immunocompetent C57Bl/6J mice inoculated with KPLG cells via tail vein injection. FIG. 10A: Representative luminescent images via in vivo imaging system (IVIS) demonstrating intrathoracic tumor growth. Recovery of tumor-derived DNA from the cytoplasmic fraction of lung alveolar macrophages in mice bearing KPLG lung adenocarcinoma. FIG. 10B: The mass of DNA recovered from the plasma and from the cytoplasmic fraction of CD45+ cells in the BALF. FIG. 10C: Size distribution of the DNA recovered from the cytoplasmic fraction of CD45+ cells in the BALF.

FIGS. 11A and 11B: Development of a detection assay for KPLG cells. FIG. 11A: Design of a sequencing-based KPLG-specific DNA test. FIG. 11B: KPLG allele fraction distribution.

FIGS. 12A and 12B: Collection of tumor-derived DNA from the cytoplasmic fraction of lung alveolar macrophages. FIG. 12A: Tumor-derived DNA is detected in the cytoplasmic part of lung alveolar macrophages collected from 10 mice inoculated with KPLG cancer cells via tail vein injection. Tumor fraction is shown for cytoplasmic fractions of alveolar macrophages collected from mice. Macrophages were either sorted with anti-CD45 staining (first 5 mice; 503, 515, 516, 519, 520) or unsorted (last 5 mice; 527, 556, 557, 558, 560).

FIG. 12B: The detection limit of cytoplasmic DNA from lung alveolar macrophages is lower than that of plasma cfDNA.

FIGS. 13A-13C: Examples of sequencing approaches capable of sequencing extremely low levels of tumor-derived DNA. FIG. 13A: Schematic illustrating procedure for obtaining blood samples from murine models for sequencing of trace tumor-derived DNA in blood. FIG. 13B: Tumor fraction can be determined with accuracy, even when as little as 1 in 100,000 DNA sequences obtained originated from a tumor. FIG. 13C Tumor-derived DNA can be accurately detected by highly sensitive next generation sequencing (NGS) methods within 1 day after administering a compound that increases the concentration of tumor-derived DNA in the blood.

### DETAILED DESCRIPTION

In various embodiments, the disclosure provides methods, compositions, and kits for leveraging tumor-associated macrophages for increasing the accessibility and/or recovery of tumor-derived DNA. The ability to obtain tumor-derived DNA directly from tumor-associated macrophages using the methods, compositions, and kits described herein represents a new strategy for gaining better access to tumor-derived DNA samples, in particular, to tumor-derived DNA samples from early stage cancers, such as early-stage lung cancers, whereby the amount of tumor-derived DNA available is low, rendering it challenging to test, detect, and otherwise evaluate such low-abundance tumor-derived DNA via standard approaches, such as detecting cell-free tumor-derived DNA present in the blood or plasma. The instant disclosure relates in part to the surprising finding that tumor-associated macrophages involved in the homeostatic clearance of apoptotic cells in a developing tumor, e.g., a lung tumor, may be leveraged as a vehicle to accumulate large amounts of tumor-derived DNA to facilitate the diagnosis of cancer, and in particular cancers that are early-stage and have otherwise low levels of cell-free tumor-derived DNA circulating in the blood.

In various embodiments, the methods, compositions, and kits described herein, involve contacting the tumor-associated macrophages involved in the homeostatic clearance of apoptotic cells with an effective amount of one or more agents which inhibit, reduce, or otherwise minimize the degradation of tumor-derived DNA by the tumor-associated macrophages. In various embodiments, the one or more agents comprises a nuclease inhibitor, which inhibits the activity of a nuclease (e.g., a DNase II) associated with the tumor-associated macrophages, which is involved in nuclease-dependent digestion of the tumor-derived DNA. In various other embodiments, the one or more agents comprises a pH-adjusting agent, which increases or lowers the pH of the microenvironment of the tumor-associated macrophages such that the nucleases (e.g., DNase II) in the tumor-associated macrophages are inhibited from degrading the tumor-derived DNA. Thus, in one aspect, the disclosure provides a method for enhancing tumor detection in a tissue, comprising administering an agent that increases the accumulation of tumor-derived DNA in a tumor-associated macrophage or population thereof, and evaluating the tumor-derived DNA, thereby detecting the tumor. In another aspect, the disclosure provides a method for increasing the recovery of tumor-derived DNA from a tumor-associated macrophage, comprising administering an agent that increases the accumulation of tumor-derived DNA in a macrophage and recovering the tumor-derived DNA from said tumor-associated macrophage. In still another aspect, the disclosure provides pharmaceutical composition for increasing tumor-derived DNA in a population of tumor-associated macrophages comprising an agent and a pharmacologically acceptable excipient, wherein the agent results in the increased accumulation of tumor-derived DNA in a tumor-associated macrophage as compared to in the absence of the agent. In yet another aspect, the disclosure provides a kit for the detection of phagocytosed DNA from a population of tumor-associated macrophages, comprising an agent for increasing phagocytosed DNA in a population of tumor-associated macrophages, and instructions for use.

### Definitions

Unless otherwise defined herein, scientific and technical terms used in connection with the present disclosure shall have the meanings that are commonly understood by those of ordinary skill in the art (e.g., the skilled artisan). The meaning and scope of the terms are clear; however, in the event of any latent ambiguity, definitions provided herein take precedent over any dictionary or extrinsic definition. Further, unless otherwise required by context, singular terms shall include pluralities and plural terms shall include the singular. In this disclosure, the use of “or” means “and/or” unless stated otherwise. Furthermore, the use of the term “including,” as well as other forms, such as “includes” and “included,” is not limiting. Also, terms such as “element” or “component” encompass both elements and components comprising one unit and elements and components that comprise more than one subunit unless specifically stated otherwise.

General terminology in cell and molecular biology can be found in “The Merck Manual of Diagnosis and Therapy”, 19th Edition, published by Merck Research Laboratories, 2006 (ISBN 0-911910-19-0); Robert S. Porter et al. (Eds.), The Encyclopedia of Molecular Biology, published by Blackwell Science Ltd., 1994 (ISBN 0-632-02182-9). General terminology definitions in molecular biology are also given in Benjamin Lewin, Genes X, published by Jones & Bartlett Publishing, 2009 (ISBN-10: 0763766321); Kendrew et al. (Eds.), Molecular Biology and Biotechnology: a Comprehensive Desk Reference, published by VCH Publishers, Inc., 1995 (ISBN 1-56081-569-8) and Current Protocols in Protein Sciences 2009, Wiley Intersciences, Coligan et al., eds.

Generally, nomenclatures used in connection with, and techniques of, cell and tissue culture, molecular biology, immunology, microbiology, genetics, and protein and nucleic acid chemistry and hybridization described herein are those well-known and commonly used in the art. The methods and techniques of the present disclosure are generally performed according to conventional methods well known in the art and as described in various general and more specific references that are cited and discussed throughout the present disclosure unless otherwise indicated. Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of subjects.

That the present disclosure may be more readily understood, select terms are defined below.

### A, an, and the

The singular forms “a,” “an,” and “the” include the plural unless the text clearly indicates otherwise. Similarly, the term “or” is intended to include “and” unless the text clearly indicates otherwise. Although methods and materials similar or equivalent to those described herein can be used in the practice or testing of the present disclosure, suitable methods and materials are described below. The abbreviation “e.g.” is derived from the Latin phrase “exempli gratia” and is used herein to give a non-limiting example. For this reason, the abbreviation “for example (e.g.)” is synonymous with the term “for example”.

### About

The terms “approximately” or “about,” as may be used interchangeably herein, and as applied to one or more values of interest, refer to a value that is similar to a stated reference value. In certain embodiments, the term “approximately” or “about” refers to a range of values that fall within 15%, 14%, 13%, 12%, 11%, 10%, 9%, 8%, 7%, 6%, 5%, 4%, 3%, 2%, 1%, or less in either direction of (i.e., percentage greater than or percentage less than) the stated reference value unless otherwise stated or otherwise evident from the context (for example, when such number would exceed 100% of a possible value).

### Cancer

The term “cancer,” as may be used herein, refers to a cell or population of cells characterized by uncontrolled proliferation. The term “tumor,” as may be used herein, refers to a contiguous population of cancer cells. A cancer may be benign, meaning that it is localized to a single tissue, or malignant, meaning that it spreads to other parts of the body through the circulatory and/or lymphatic system. A cell or population of cells may be “pre-cancerous,” meaning that they share some characteristics of a cancer and risk developing into a cancer. Cells may become cancerous as a result of accumulated mutations in their genome. Examples of cancer include but are not limited to colorectal cancer, lung cancer, breast cancer, pancreatic cancer, prostate cancer, bladder cancer, kidney cancer, thyroid cancer, uterine cancer, cervical cancer, ovarian cancer, testicular cancer, esophageal cancer, stomach cancer, liver cancer, brain cancer, peritoneal cancer, lymphoma, leukemia, multiple myeloma, neuroblastoma, osteosarcoma, and soft tissue sarcoma.

### Cell-Free DNA

The terms “cell-free DNA” or “cfDNA,” as may be used interchangeably herein, refer to deoxyribonucleic acid species that occur extracellularly. Cell-free DNA may originate from one or more cells. Cell-free DNA may originate from one or more cell types. Cell-free DNA may originate from healthy cells or diseased cells. Cell-free DNA may be single-stranded or double stranded. Cell-free DNA may interact with other species, such as histone proteins, to form higher order structures, such as nucleosomes. In some embodiments, cell-free DNA originates from the cells of a subject. In some embodiments, cell-free DNA originates from both healthy and diseased cells of a subject. In some embodiments, cell-free DNA encodes one or more genes belonging to the subject's genome. In some embodiments, cell-free DNA contains mutations that are indicative of a disease, such as a cancer.

### Decrease

The terms “decrease,” “reduced,” “reduction,” or “inhibit” are all generally statistically significant herein. The terms are used to indicate a decrease in quantity, concentration, level, or the like. However, to prevent misunderstandings, “decreased,” “decreasing,” “decreasing,” or “inhibiting” is a reduction of at least 10% compared to the reference level, e.g., at least about 20% compared to the reference level. The % reduction may also be at least about 20%, or at least about 30%, or at least about 40%, or at least about 50%, or at least about 60%, or at least about 70%, or at least about 80%, or at least about 90%, or up to 100% reduction compared to the reference level. The reduction level may also be expressed in terms of fold-reduction, and includes at least a 2-fold reduction, or at least a 3-fold reduction, or at least a 3-fold reduction, or at least a 3-fold reduction, or at least a 3-fold reduction, or at least a 4-fold reduction, or at least a 5-fold reduction, or at least a 6-fold reduction, or at least a 7-fold reduction, or at least a 8-fold reduction, or at least a 9-fold reduction, or at least a 10-fold reduction, or at least a 11-fold reduction, or at least a 12-fold reduction, or at least a 13-fold reduction, or at least a 14-fold reduction, or at least a 15-fold reduction, or at least a 16-fold reduction, or at least a 17-fold reduction, or at least a 18-fold reduction, or at least a 19-fold reduction, or at least a 20-fold reduction, or at least a 25-fold reduction, or at least a 50-fold reduction, or more.

### Increase

The terms “increased,” “increase,” “enhance” or “activate” are all generally statistically significant herein. The terms are used to indicate an increase in quantity, concentration, level, or the like. To prevent misunderstanding, the terms “increased,” “increase,” “enhance,” or “activate” is increase of at least 10% compared to the reference level, e.g., at least about 20% compared to the reference level. The % increase may also be at least about 20%, or at least about 30%, or at least about 40%, or at least about 50%, or at least about 60%, or at least about 70%, or at least about 80%, or at least about 90%, or up to 100% increase compared to the reference level. The increase level may also be expressed in terms of fold-increase, and includes at least a 2-fold increase, or at least a 3-fold increase, or at least a 3-fold increase, or at least a 3-fold increase, or at least a 3-fold increase, or at least a 4-fold increase, or at least a 5-fold increase, or at least a 6-fold increase, or at least a 7-fold increase, or at least a 8-fold increase, or at least a 9-fold increase, or at least a 10-fold increase, or at least a 11-fold increase, or at least a 12-fold increase, or at least a 13-fold increase, or at least a 14-fold increase, or at least a 15-fold increase, or at least a 16-fold increase, or at least a 17-fold increase, or at least a 18-fold increase, or at least a 19-fold increase, or at least a 20-fold increase, or at least a 25-fold increase, or at least a 50-fold increase, or more.

### Isolated

The terms “isolated” or “partially purified” as used herein (e.g., in the context of isolated cfDNA) refers to a biological material (e.g., a cfDNA) that has been separated from other biological materials, e.g., from a biological sample of blood or other fluid.

### Biological Sample

The term “biological sample” as used herein generally refers to a tissue or body fluid sample derived from a subject. Biological samples can be obtained directly from a subject. The biological sample can be or comprise one or more nucleic acid molecules, such as deoxyribonucleic acid (DNA) or ribonucleic acid (RNA) molecules (e.g., cfDNA). The biological sample may be derived from any organ, tissue or biological fluid. The biological sample may comprise, for example, biological fluids or solid tissue samples. An example of a solid tissue sample is a tumor sample, e.g., a solid tumor biopsy. Biological fluids include, for example, blood, serum, plasma, tumor cells, saliva, urine, lymphatic fluid, synovial fluid, interstitial fluid, cerebrospinal fluid, prostate fluid, semen, sputum, mucus, gastric acid, bile, feces, tears, and derivatives thereof.

### Liquid Biopsy

The term “liquid biopsy” as used herein generally refers to a non-invasive or minimally invasive laboratory test or assay (e.g., of a biological sample or of a cell-free DNA). Such a “liquid biopsy” assay may report a measurement of one or more tumor-associated marker genes (e.g., minor allele frequency, gene expression, or protein expression). For example, a circular tumor DNA test from Guardant Health, a Spotlight 59 oncology panel from Fluxion Biosciences, an Ultrasik from Agena Bioscience, Such as the UltraSEEK lung cancer panel, the Foundation ACT fluid biopsy assay from Foundation Medicine, and the PlasmaSELECT assay from Personal Genome Diagnostics, are commercially available have. Such assays may report a measure of the minor allele fraction (MAF) value for each set of genetic variants (e.g., single nucleotide variation (SNV), copy number variation (CNV), insertion/deletion (Indel), and/or fusion). The methods and compositions described herein for boosting the levels of cfDNA may be used in combination with a liquid biopsy to assay for the presence of a disease marker. In some embodiments, the liquid biopsy involves a non-invasive or minimally invasive laboratory test or assay on a sample of blood. In such cases, the liquid biopsy can be referred to as a “blood biopsy.”

### Mutation

The term “mutation,” as may be used herein, refers to a change, alteration, or modification to a nucleotide in a nucleic acid (e.g., a cfDNA) as compared to its wild-type sequence. For example, without limitation, mutations may include substitutions, insertions, deletions, or any combination of the same in the cfDNA which is concentrated and then assayed by the methods described herein. In some embodiments, there is at least one mutation in herein disclosed cfDNA. In some embodiments, there is more than one mutation. In some embodiments, where there is more than one mutation, the mutations are distinct (e.g., not of the same type, e.g., substitutions, insertions, deletions). In some embodiments, where there is more than one mutation, the mutations are the same (e.g., the same type, e.g., substitutions, insertions, deletions). In some embodiments, the mutations result in a shifted reading frame (frameshift). In some embodiments, the mutations are indicative of a disease, such as a cancer. In other embodiments, the mutations are “disease-associated” mutations, which refers to mutations that predict that the subject in which the mutation exists has an increased chance or risk of having or developing a disease, e.g., cancer. Such disease-associate mutations may include point mutations wherein a single base is added, deleted or changed in a subject's DNA or RNA. The mutations may also be nonsense, missense, or silent mutations. A nonsense mutation occurs when one nucleotide is substituted and this leads to the formation of a stop codon instead of a codon that encodes an amino acid. A stop codon is a certain sequence of bases (TAG, TAA, or TGA in DNA, and UAG, UAA, or UGA in RNA) that terminates the production of an amino acid chain during translation. Stop codons are always found at the end of a mRNA sequence when a protein is being produced, but if a substitution causes one to appear in another place, it will prematurely terminate the amino acid sequence and prevent the correct protein from being produced. Like a nonsense mutation, a missense mutation occurs when one nucleotide is substituted and a different codon is formed, but in this case the new codon encodes a different amino acid than was originally encoded. For example, if a missense substitution changes a codon from AAG to AGG, the amino acid arginine will be produced during translation instead of lysine. A missense mutation is considered conservative if the amino acid formed via the mutation has similar properties to the one that was originally encoded. It is non-conservative if the amino acid has different properties, which can disrupt the structure and function of a protein. A silent mutation is one in which a nucleotide is substituted but the mutation does not change the amino acid that a codon encodes. This can occur because multiple codons can code for the same amino acid. For example, AAG and AAA both code for lysine, so if the G is mutated to A, the same amino acid will be coded for during translation and the protein will not be affected. Even so, silent mutations can have deleterious effects, e.g. by altering mRNA stability. Disease-associated mutations may also include mutations that change the copy number of a gene by either duplicating or removing all or part of a gene in the genome. Disease-associated mutations may also include epigenetic modifications, such as changes in DNA methylation. DNA methylation refers to the addition of a methyl group to certain bases (C or A) in a nucleic acid molecule (e.g., changing C to 5-methylcytosine, mC). Changes in DNA methylation can occur within a coding portion of a gene (part of a gene that is transcribed and translated into protein) or within a non-coding portion of a gene (part of a gene that is transcribed and not translated into protein). Changes in DNA methylation can occur within the promotor sequence of a gene. Changes in the DNA methylation pattern of a gene can its expression level (i.e. cause it to be expressed at a higher or lower level than it is typically expressed).

### Substantially

The term “substantially,” as may be used herein, when used to describe the degree or abundance of an activity, generally refers to the value of the activity as being an amount which is achievable without undue effort. As can be appreciated, this amount may vary depending on the activity being performed, with simpler activities requiring a higher threshold and more complex activities requiring a lower threshold.

### Subject

As used herein, “subject” means a human or animal. Usually, the animal is a vertebrate such as a primate, rodent, livestock animal, or hunting animal. Primates include chimpanzees, cynomolgus monkeys, spider monkeys, and macaques such as rhesus monkeys. Rodents include mice, rats, hamsters, rabbits, guinea pigs, squirrels, woodchucks, ferrets. Livestock and game animals include cattle, horses, pigs, deer, bison, buffalo, cat species such as domesticated cats, dog species such as domesticated dogs, foxes, wolves, birds such as chickens, turkeys, ducks, geese, emus, ostriches, and fish such as trout, catfish, and salmon. In some embodiments, the subject is a mammal, such as a primate, such as a human. The terms “individual,” “patient” and “subject” are used interchangeably herein. Preferably, the subject is a mammal. The mammal can be a human, non-human primate, mouse, rat, dog, cat, horse, or cow, but is not limited to these examples. Mammals other than humans can be conveniently used, for example, as subjects that represent animal models of cancer, e.g., a particular type of cancer, such as, lung cancer. The subject can be male or female. In various embodiment, the subject is a patient that has or is at risk of having a disease state, such as cancer, and is in need to being evaluated, e.g., by a liquid biopsy, to test for the risk of having or developing a disease, e.g., cancer. In other embodiments, that subject is a patient that has already been diagnosed or identified as having or having a disease in need of treatment (e.g., cancer), or one or more complications associated with such diseases. In other embodiments, a subject is a patient that has already been treated for a disease (e.g., cancer) or one or more complications associated with a disease, such as cancer. Alternatively, a subject can also be a patient that has not been previously diagnosed as having a disease (e.g., cancer) or one or more complications associated with the disease. For example, a subject can be a patient that exhibits one or more risk factors for a disease, or one or more complications associated with the disease (e.g., cancer), or a patient that does not exhibit a risk factor.

### Subject in Need

A “subject in need” of a diagnosis and/or treatment for a particular condition (e.g., cancer) can be a subject who has a condition, has been diagnosed with a condition, or is at risk of developing the condition.

### Agent

As used herein, the term “agent” refers to any material which is capable of causing a specific outcome when applied to a biological setting. An agent can be a naturally occurring, semi-synthetic, or fully synthetic chemical compound. An agent can facilitate or interfere with one or more chemical reactions occurring in an organism, thereby having one or more biological effects. An agent having a biological effect is said to be bioactive. An agent may be administered to an organism with the intent of producing a desired biological effect. An agent may be administered to an organism in the presence of additional materials having no discernable biological effect, such as an excipient compound.

### Effective Amount

As used herein, the term “effective amount” refers to the amount of an administered agent that is sufficient to produce an intended biological effect in an organism that the agent is administered to. “Effective amount” is synonymous with the terms “effective dose” and “effective concentration”.

### Phagocytes and Macrophages

The term “phagocyte” (i.e., phagocytic cell) refers to any cell that phagocytoses other cells. The term “macrophage” refers to a specific type of phagocyte. Macrophages are a type of differentiated leukocyte (i.e., white blood cell) that is primarily responsible for the uptake and clearance of, for example, foreign cells such as microorganisms, apoptotic cells, and cellular debris. Macrophages may be found circulating in the blood or localized to specific locations of the host body, such as terminal alveoli in the lungs. Macrophages may be classically activated macrophages (M1) or alternatively activated macrophages (M2).

### DNA Degradation

The term “DNA degradation” is used to refer to the various processes by which DNA species are hydrolyzed into smaller DNA fragments and/or individual nucleotides. DNA degradation can occur as a result of enzymatic processes, such as those that are catalyzed by deoxyribonucleases (i.e., DNases). DNA degradation can occur in a cell free (extracellular) environment, within the cytosol of a cell, or within one or more membrane-enclosed compartments (organelles) of a cell.

### Small Molecule

The term “small molecule,” as used herein, refers to molecule having a relatively low molecular weight. A small molecule may have a molecular weight of less than 500 daltons, less than 600 daltons, less than 700 daltons, less than 800 daltons, less than 900 daltons, or less than 1 kilodalton. A small molecule may diffuse freely across a cell membrane. A small molecule may be an effector that modulates the function of a macromolecule, such as a protein. A small molecule may be an agonist or an inhibitor of an enzyme.

### Expression

The term “expression” is broadly used to refer to the processes by which one or more genes in a cell are transcribed by RNA polymerases to produce RNA transcripts which may be translated by ribosomes to produce one or more proteins. Expression may refer to either or both of the acts of transcribing genes to RNA and translating RNA into protein. Expression may be described in absolute terms, such as the number of a particular type of RNA transcript or protein present in a cell at a given time. Expression may also be described in terms that are relative, such as when cells are treated with one or more compounds that causes expression of one or more genes and/or proteins to increase or decrease.

### Activity

The term “activity,” as is used herein, refers to the rate at which an enzyme catalyzes a particular chemical reaction. Activity may be described in absolute terms, such as the number of reactions an enzyme catalyzes on average per second. Activity may also be described in terms that are relative, such as when cells are treated with one or more compounds that causes the activity of one or more enzymes to increase or decrease.

### Microsphere

The term “microsphere” refers to an approximately spherical microstructure that is invisible to the naked eye, having a diameter across its longest axis of less than 0.5 μm, less than 0.6 μm, less than 0.7 μm, less than 0.8 μm, less than 0.9 μm, less than 1 μm, less than 2 μm, less than 3 μm, less than 4 μm, less than 5 μm, less than 6 μm, less than 7 μm, less than 8 μm, less than 9 μm, less than 10 μm, less than 20 μm, less than 30 μm, less than 40 μm, less than 50 μm, less than 60 μm, less than 70 μm, less than 80 μm, less than 90 μm, or less than 100 μm. A microsphere may be hollow, being composed of a thin wall enclosing a relatively large interior void. A microsphere may be composed of any pharmacologically acceptable material capable of forming such a structural matrix. A microsphere may be porous or perforated. Microspheres having any or all of these properties may be produced by spray drying a liquid feed stock to produce a dry powder. A powder of porous microspheres may have a relatively low bulk density of approximately 0.5 g/cm³ or less, wherein the powder exhibits reduced van der Waals attractive forces compared to a relatively nonporous powder of the same composition. A microsphere may be composed of a material capable of adhering to a biologically active agent. A microsphere may be aerosolized, such that it can be administered to a subject through inhalation, especially for the purpose of delivering a bound agent.

### Routes of Administration

An agent may be delivered to a subject by one or more routes of administration. An agent may be enterally administered through the gastrointestinal tract, parenterally administered through any non-enteral route of administration, or topically administered to an external surface of the subject. Parenteral administration can be performed by injection of an agent into a specific location, tissue, or organ of a subject (e.g., intradermal, intravenous, subcutaneous, or intramuscular injection). Alternatively, parenteral administration can be inhalational, wherein the agent is administered by oral and/or nasal inhalation for uptake in the respiratory tract.

### Pharmaceutical Composition

The term “pharmaceutical composition” refers to any composition or formulation that is suitable for administration to a subject. A pharmaceutical composition or a component thereof is said to be “pharmaceutically acceptable” if it is generally safe and non-toxic when administered to a subject.

### Tumor DNA and Efferocytosis

The methods, compositions, and kits described herein are based, at least in part on the previously unrecognized utility of a subject's own macrophages as a means of collecting tumor-derived DNA for subsequent recovery and analysis. These techniques substantially enhance current capabilities for assaying, detecting, or otherwise evaluating tumor DNA in subjects who are suspected of having, known to have, or known to have had cancer.

Cancer cells within tumors exhibit an accelerated rate of proliferation compared to cells of healthy tissues, but even so many cells within a growing tumor ultimately undergo apoptosis, or programmed cell death, just as healthy cells do. The process by which apoptotic tumor cells are cleared is generally identical to that by which non-cancerous cells are cleared. Efferocytosis is the phenomenon by which cells in various stages of apoptosis are regularly engulfed and removed by subjects' phagocytic cells (phagocytes). Efferocytosis can be mediated by several different phagocytic cell types. Efferocytosis primarily occurs by way of macrophages or dendritic cells, cell types whose primary purpose is to phagocytose other cells, whether these be foreign cell types or a host's own cells. Alternately, efferocytosis can be mediated by certain other cells, such as epithelial cells or fibroblasts, which generally perform other functions in a subject by can be compelled to phagocytose apoptotic cells. Efferocytosis occurs in four major steps. First, apoptotic cells must secrete a chemotactic gradient to attract phagocytes. Major chemoattractants include CX3CL1, sphingosine-1-phosphate (S1P), lysophosphatidylcholine (LPC), and free nucleotides. Second, apoptotic cells must additionally secrete or present on their outwardly facing cell surface one or more chemicals that promote engulfment by a phagocyte, such as phosphatidylserine (PS). Third, the phagocyte must recognize the apoptotic cell. Recognition may occur, for instance, through PS receptors on its surface. Finally, the phagocyte engulfs and internalizes the apoptotic cell, placing it into a membrane-enclosed intracellular body (phagosome) which is subsequently fused with the lysosome, resulting in hydrolysis of various components of the cell, including its membranes, proteins, and nucleic acids. To reduce inflammation caused by the release of pro-inflammatory species that are produced during hydrolysis of the phagocytosed cell, such as aggregated DNA fragments produced during degradation of the apoptotic cell's DNA, phagocytes may also secrete anti-inflammatory cytokines, such as IL-10 and TGFβ. Normal functioning of efferocytosis is a necessity for the maintenance of organized growth, wound healing, and homeostasis in a subject, but is also important for the clearance of cancer cells that have or are undergoing apoptosis.

Complete clearance of phagocytosed cells during efferocytosis requires the activity of various enzymes localized to the phagocytic lysosomes, such as nucleases, phosphatases, proteases, esterases, phospholipidases, and various polysaccharide hydrolases. Collectively, these enzymes contribute broadly to the breakdown of various macromolecules within engulfed apoptotic cell, including DNA (including tumor-derived DNA), RNA, proteins, lipids, and polysaccharides. The majority of these lysosomal enzymes function optimally at an acidic pH and are therefore inactive or minimally active outside of lysosomes or when the pH of lysosomes is transiently increased.

Without desiring to be bound by theory, of particular importance to the present disclosure are enzymes that catalyze the degradation of phagocytosed cell DNA. The genomes of phagocytosed cells, in addition to apoptotic fragments produced thereof, are potentially toxic to phagocytes and must be recycled back into free nucleotides. Apoptotic cells begin to degrade their genomes into nucleosomal fragments via enzymes such as caspase-activated DNase (CAD), however deoxyribonucleases (DNases) of the phagocyte are required to complete DNA degradation. This process is largely mediated by DNase II (EC 3.1.22.1), also known variably as pancreatic DNase II, deoxyribonucleate 3′-nucleotidohydrolase, and acid DNase. DNase II is an essential mammalian endonuclease that is required for a variety of cellular processes pertaining to the efficient removal of DNA, including the maturation of red blood cells during erythropoiesis. Several isoforms of DNase II are expressed by humans, most notably DNase II alpha, which is the ubiquitously expressed isoform, as well as DNase II beta, which is a tissue-specific isoform of DNase II (also known as DNase II-like acid DNase, or DLAD), which is required to degrade DNA in the cornea. DNase II functions optimally at low pH, e.g., at a pH of about 4.5 to about 5.5.

DNase II is thought to function in three distinct phases. First, DNase II binds to double stranded DNA and nicks the deoxyribose-phosphate backbone of these duplexes, primarily introducing single strand breaks while releasing free phosphate. Second, DNase II cleaves the nicked DNA through a mechanism of action requiring specific histidine residues (His115, His132, and His 297), producing free nucleotides as well as a cleavage-resistant 3′ tetranucleotide fragment. Finally, as the DNA duplex is increasingly degraded a hyperchromatic shift occurs as it denatures into single strands, each of which is then degraded separately.

Just as DNase II is required to efficiently degrade the genomes of healthy phagocytosed cells, so too is it required for the efficient degradation of phagocytosed tumor cells. For this reason, temporary inhibition of DNase II, whether directly or by increasing the pH of phagocyte lysosomes, results in the transient accumulation of DNA within phagocyte lysosomes. In this way, phagocytes, such as a subject's own macrophages, for instance, can be used as a microscopic vessel for the collection of any tumor-derived DNA present in a subject. This DNA can then be collected for analysis from the subject in a quantity that would otherwise be impossible or impractical by other methods presently available.

### Lung Tumor DNA

Detection of certain cancer types could particularly benefit from the disclosed methods, particularly those for which detection by other methods is invasive, prone to high false positive or false negative rates, and/or generally unsuccessful except in cases where a cancer has already progressed considerably. One set of cancers which could especially benefit from the methods disclosed herein are lung cancers.

Lung cancer is the leading cause of cancer-related mortality, with >1.7 million annual deaths worldwide. Survival is strongly associated with stage at diagnosis (five-year overall survival: 56% in localized, 29% in regional, and 5% in distant-stage disease) and yet, fewer than 20% of lung cancers are diagnosed early (i.e., during Stage I). Lung cancer screening via low-dose computed tomography (CT) has proven to reduce lung cancer mortality in high-risk patients. However, many patients who are ultimately diagnosed with lung cancer do not initially fit current chest CT screening criteria. Furthermore, there is an increasing number of incidental nodules being identified during imaging studies obtained for alternative indications. This presents major diagnostic dilemmas, including how to better identify patients at risk for lung cancer and what to do about pulmonary nodules once they are discovered. Currently, the only ways to tell which pulmonary nodules are cancerous are to monitor the nodules for cancerous growth or to perform an invasive procedure such as a biopsy. Each harbors risk, either for delaying diagnosis or for potentially causing unnecessary harm. Even when a biopsy is performed, it remains exceedingly difficult to accurately biopsy peripheral nodules due to their small size, despite these nodules being where 70% of lung cancers develop. With >2.1 million new lung cancer cases being diagnosed worldwide each year, better lung cancer diagnostics are urgently needed.

Lung cancer is traditionally divided into two major types, non-small cell lung cancer (NSCLC) and small cell lung cancer (SCLC). NSCLC is more common, with lung adenocarcinoma being the most common subtype of NSCLC. Lung adenocarcinomas typically arise in the distal airways or in the terminal alveoli, which are challenging to biopsy. Bronchoscopy cannot reach terminal alveoli, while bronchoalveolar lavage (BAL; where fluid is instilled and then removed using a bronchoscope) has limited sensitivity in this setting, with BAL fluid (BALF) cytology having only 29% sensitivity for malignancy. With transbronchial needle aspiration (TBNA), the sensitivity for peripheral lung cancers can be increased to 50-60%. Recent studies have investigated genomic testing for tumor DNA in BALF to improve accuracy of diagnosis. Yet, while tumor DNA can be recoverable in BALF, the yield in early stage lung cancer is very limited, likely due to the relatively low tumor burden and local clearance any tumor DNA.

As in other cancer types, significant numbers of lung cancer cells undergo apoptosis in a developing tumor, producing distinctive DNA signatures that enable cancer detection. The vast majority of tumor-derived DNA is cleared via efferocytosis of apoptotic cells or apoptotic bodies in the airway lumen, where lung tumors develop and interact with alveolar macrophages. Due to the close proximity between developing tumors and alveolar macrophages, blockage of lysosomal DNases within alveolar macrophages, e.g., inhibition of DNase II, is uniquely positioned to preserve DNA derived from apoptotic lung cancer cells for subsequent recovery and detection.

### Agents for Inhibiting Nuclease-Based Degradation of Tumor DNA

In one aspect, the present disclosure relates to the administration to a subject of an effective dose of an agent capable of inhibiting the deoxyribonuclease-based degradation of tumor-derived DNA. In some embodiments, an agent is capable of inhibiting the deoxyribonuclease-based degradation of cell free tumor-derived DNA. In other embodiments, an agent is capable of inhibiting the deoxyribonuclease-based degradation of tumor-derived DNA within one or more phagocytic cell types, such as alveolar macrophages in the case of lung cancer. In further embodiments, an agent is capable of inhibiting the deoxyribonuclease-based degradation of tumor-derived DNA within a specific membrane-enclosed organelle of a phagocytic cell type, such as a lysosome.

In some embodiments, an agent capable of inhibiting deoxyribonuclease-based degradation of tumor DNA increases the concentration of tumor-derived DNA by up to 2-fold, up to 3-fold, up to 4-fold, up to 5-fold, up to 10-fold, up to 25-fold, up to 50-fold, up to 100-fold, up to 200-fold, up to 300-fold, up to 400-fold, up to 500-fold, up to 600-fold, up to 700-fold, up to 800-fold, up to 900-fold, or up to 1000-fold. The concentration of tumor-derived DNA may be increased, for instance, within lysosomes of a macrophage or a population of macrophages.

In some embodiments, an agent capable of inhibiting deoxyribonuclease-based degradation of tumor DNA reduces the rate of tumor-derived DNA degradation by up to 5%, up to 10%, up to 15%, up to 20%, up to 25%, up to 30%, up to 35%, up to 40%, up to 45%, up to 50%, up to 55%, up to 60%, up to 65%, up to 70%, up to 75%, up to 80%, up to 85%, up to 90%, up to 95%, up to 99%, or up to 100%, as compared to the rate of tumor-derived DNA degradation in the absence of the agent. The rate of tumor-derived DNA degradation may be reduced, for instance, within lysosomes of a macrophage or a population of macrophages.

In some embodiments, an agent that is capable of inhibiting deoxyribonuclease-based degradation of tumor-derived DNA acts systemically. In other embodiments, an agent that is capable of inhibiting nuclease-based degradation of tumor-derived DNA acts only within a localized region of a subject, such as within a specific organ, tissue, or population of cells.

In some embodiments, an agent capable of inhibiting the deoxyribonuclease-based degradation of cell free tumor-derived DNA is a small molecule inhibitor. In some embodiments, an agent capable of inhibiting the deoxyribonuclease-based degradation of cell free tumor-derived DNA is a peptide inhibitor. In some embodiments, an agent capable of inhibiting the deoxyribonuclease-based degradation of cell free tumor-derived DNA is a small molecule or peptide inhibitor of DNase II, examples of which are well known within the art (e.g., Sperinde et al. “Phage display selection of a peptide DNase II inhibitor that enhances gene delivery” J Gene Med. 2001 March-April; 3(2):101-8, which are herein incorporated by reference).

In some embodiments, an agent capable of inhibiting the deoxyribonuclease-based degradation of cell free tumor-derived DNA is an oligonucleotide capable of interfering with expression of one or more deoxyribonucleases. In some embodiments, an agent capable of interfering with expression of one or more deoxyribonucleases is a small interfering RNA (siRNA), short hairpin RNA (shRNA), or micro RNA (miRNA). In some embodiments, an agent capable of interfering with expression of one or more deoxyribonucleases is an oligonucleotide capable of interfering with expression of DNase II, examples of which are commercially available (e.g., Santa Cruz Biotechnology #sc-41507, Santa Cruz Biotechnology #sc-41507-SH, Sigma-Aldrich #NM_001375, Sigma-Aldrich #SHCLND-NM_001375) or are otherwise well known within the art (e.g., Barker and Diamond. “DNase II Knockdown in Human Endothelial Cells Does Not Improve Non-Viral Transfection Efficiency.” Molecular Therapy 13 (2006): S395; Ding et al. “Oxidant stress in mitochondrial DNA damage, autophagy and inflammation in atherosclerosis.” Scientific reports 3.1 (2013): 1-6; Ding, et al. “LOX-1, mtDNA damage, and NLRP3 inflammasome activation in macrophages: implications in atherogenesis.” Cardiovascular research 103.4 (2014): 619-628; and Rodero et al. “Type I interferon-mediated autoinflammation due to DNase II deficiency.” Nature communications 8.1 (2017): 1-15, each of which are incorporated by reference herein).

### Agents for Modifying Phagocyte Microenvironment to Limit DNA Degradation

In another aspect, the present disclosure relates to the administration to a subject of an effective dose of an agent capable of modifying the microenvironment in which one or more deoxyribonucleases functions so as to limit the degradation of tumor-derived DNA. In some embodiments, an agent is capable of modifying the microenvironment to limit degradation of cell free tumor DNA. In other embodiments, an agent is capable of modifying the microenvironment to limit degradation of tumor-derived DNA within one or more phagocytic cell types, such as alveolar macrophages. In further embodiments, an agent is capable of modifying the microenvironment to limit degradation of tumor-derived DNA within a specific membrane-enclosed organelle of a phagocytic cell type, such as a lysosome.

In some embodiments, an agent capable of modifying the microenvironment to limit degradation of tumor DNA increases the concentration of tumor DNA by up to 2-fold, up to 3-fold, up to 4-fold, up to 5-fold, up to 10-fold, up to 25-fold, up to 50-fold, up to 100-fold, up to 200-fold, up to 300-fold, up to 400-fold, up to 500-fold, up to 600-fold, up to 700-fold, up to 800-fold, up to 900-fold, or up to 1000-fold. The concentration of tumor DNA may be increased, for instance, within the lysosomes of a macrophage or a population of macrophages.

In some embodiments, an agent capable of modifying the microenvironment to limit degradation of tumor DNA reduces the rate of tumor DNA degradation by up to 5%, up to 10%, up to 15%, up to 20%, up to 25%, up to 30%, up to 35%, up to 40%, up to 45%, up to 50%, up to 55%, up to 60%, up to 65%, up to 70%, up to 75%, up to 80%, up to 85%, up to 90%, up to 95%, up to 99%, or up to 100%, as compared to the rate of tumor DNA degradation in the absence of the agent. The rate of tumor DNA degradation may be reduced, for instance, within the lysosomes of a macrophage or a population of macrophages.

In some embodiments, an agent that is capable of modifying the microenvironment to limit degradation of tumor DNA acts systemically. In other embodiments, an agent that is capable of modifying the microenvironment to limit degradation of tumor DNA acts only within a localized region of a subject, such as within a specific organ, tissue, or population of cells.

In some embodiments, an agent capable of modifying the microenvironment to limit degradation of tumor DNA is a small molecule. In some embodiments, an agent capable of modifying the microenvironment to limit degradation of tumor DNA results in an increase in the pH of lysosomes when administered to a subject. In some embodiments, an agent capable of modifying the microenvironment to limit degradation of tumor DNA prevents the fusion of phagocytic vesicles with lysosomes when administered to a subject. In some embodiments, an agent capable of modifying the microenvironment to limit degradation of tumor DNA is chloroquine or a derivative thereof, such as hydroxychloroquine, examples of which are well known within the art (e.g., Mauthe et al. “Chloroquine inhibits autophagic flux by decreasing autophagosome-lysosome fusion.” Autophagy 14.8 (2018): 1435-1455; and Chen et al. “Chloroquine modulates antitumor immune response by resetting tumor-associated macrophages toward M1 phenotype.” Nature communications 9.1 (2018): 1-15, which are incorporated by reference herein). In some embodiments, an agent capable of modifying the microenvironment to limit degradation of tumor DNA is a lysosomal inhibitor not structurally related to chloroquine, such as bafilomycin or BRD1240, examples of which are well known within the art (e.g., Bonam et al. “Lysosomes as a therapeutic target” Nat Rev Drug Discov (2019) 18, 923-948, which is incorporated by reference herein). In some embodiments, an agent capable of modifying the microenvironment to limit degradation of tumor DNA is an inhibitor of one or more lysosomal proton pumps, examples of which are well known within the art (e.g., Liu et al. “Inhibition of lysosomal enzyme activities by proton pump inhibitors”. J Gastroenterol. 2013 December; 48(12):1343-52, which is incorporated by reference herein).

### Agents for Enhancing Phagocyte Recovery

In another aspect, the present disclosure relates to the administration to a subject of an effective dose of an agent capable of enhancing the ability of phagocytes to be collected in a biological sample. In some embodiments, an agent can reduce the adherence of one or more phagocytic cell types, such as alveolar macrophages, thereby increasing their tendency to be collected, for example, in a biological liquid. In some embodiments, an agent capable of reducing the adherence of one or more phagocytic cell types indirectly enhances the amount of tumor-derived DNA that can be collected from phagocytes, by enhancing the quantity of phagocytes that may be collected.

### Biological Samples and Techniques for Obtaining Same

Techniques for increasing the tumor DNA concentration within phagocytes, such as macrophages, may further involve a step of collecting phagocytes in a biological sample. A biological sample containing phagocytes may involve the collection of one or more biological fluids thought to contain phagocytes. Examples of biological fluids include, but are not limited to, blood, plasma, serum, lymph, synovial fluid, interstitial fluid, cerebrospinal fluid, urine, mucus, and saliva, methods of collection for which are well known in the art. Obtaining a biological sample containing phagocytes may involve deriving a biological sample from another biological sample or biological fluid, such as, for instance, deriving a fraction of blood from whole blood.

The methods and compositions described herein for boosting the concentration of tumor DNA in phagocytes may be used in combination with a liquid biopsy to assay for the presence of a disease marker. In some embodiments, the liquid biopsy involves a non-invasive or minimally invasive laboratory test or assay on a sample of blood. In such cases, the liquid biopsy can be referred to as a “blood biopsy.”

Techniques for increasing the tumor DNA concentration within macrophages of the lungs, such as alveolar macrophages, may further involve a step of collecting macrophages in a biological sample from the lungs. A biological sample containing macrophages from the lungs may be collected from peripheral lung tissue, such as terminal alveoli. A biological sample containing macrophages from the lungs may be collected through the use of non-invasive techniques.

A biological sample containing macrophages from the lungs may be collected, for instance, as a sputum sample. A sputum sample may be collected by means of sputum induction, methods for which are well known in the art (e.g., Paggiaro et al. “Sputum induction” Eur Respir J Suppl. 2002 September; 37:3s-8s, which is incorporated by reference herein).

A biological sample containing macrophages from the lungs may also be collected, for instance, as bronchoalveolar lavage (BAL) fluid. BAL fluid may be collected by means of BAL, methods for which are well known in the art (e.g., Anzueto et al. “The technique of bronchoalveolar lavage. A guide to sampling the terminal airways and alveolar space”. J Crit Illn. 1992 November; 7(11):1817-24; and Poletti et al. “Bronchoalveolar lavage in malignancy”. Semin Respir Crit Care Med. 2007 October; 28(5):534-45, which are incorporated by reference herein).

The methods, compositions, kits, and principles described herein may be used not only with phagocytic macrophages associated with tumors (e.g., lung cancer), but associated with other diseases and conditions anywhere in the body that involve the phagocytic ingestion of disease cells or tissues which result in the accumulation of DNA from a diseased tissue or cell inside the phagocytic macrophage.

For example, the methods, compositions, kits and principles described herein in the cancer context, may be utilized to collect DNA from cells or tissues involved in an autoimmune or immune related disease or condition. As used herein, the term “autoimmune or immune related disease or condition” refers to any disease or condition that adversely affects the functioning of the immune system. Examples of autoimmune or immune related diseases or conditions include, but are not limited to, antiphospholipid antibody syndrome, systemic lupus erythematosus, rheumatoid arthritis, autoimmune vasculitis, celiac disease, autoimmune thyroiditis, blood transfusion Postimmunization, maternal-fetal incompatibility, blood transfusion reaction, immune deficiency such as IgA deficiency, unclassifiable immunodeficiency, drug-induced lupus, diabetes, type I diabetes, type II diabetes, juvenile onset diabetes, juvenile rheumatism Osteoarthritis, psoriatic arthritis, multiple sclerosis, immunodeficiency, allergy, asthma, psoriasis, atopic dermatitis, allergic contact dermatitis, chronic skin disease, amyotrophic lateral sclerosis, chemotherapy-induced injury, Graft-versus-host disease, bone marrow transplant rejection, ankylosing spondylitis, atopic eczema, pemphigus, Behcet's disease, chronic fatigue syndrome fibromyalgia, induction of chemotherapy Injury, myasthenia gravis, glomerulonephritis, allergic retinitis, systemic sclerosis, subacute cutaneous lupus erythematosus, cutaneous lupus erythematosus, sjogren's syndrome, autoimmune nephritis, autoimmune vasculitis, autoimmunity Hepatitis, autoimmune carditis, autoimmune encephalitis, autoimmune-mediated blood disease, lc-SSc (localized scleroderma scleroderma), dc-SSc (diffuse scleroderma), autoimmunity Thyroiditis (AT), Graves' disease (GD), myasthenia gravis, multiple sclerosis (MS), ankylosing spondylitis, transplant rejection, immunosenescence, rheumatic/autoimmune disease, mixed connective tissue Disease, spondyloarthritis, psoriasis, psoriatic arthritis, myositis, scleroderma, dermatomyositis, autoimmune vasculitis, mixed connective tissue disease, idiopathic thrombocytopenic purpura, Crohn's disease, human aju Disease, osteoarthritis, juvenile chronic arthritis, spondyloarthropathy, idiopathic inflammatory myopathy, systemic vasculitis, sarcoidosis, autoimmune hemolytic anemia, autoimmune thrombocytopenia, thyroiditis, immune-mediated Kidney disease, central or peripheral demyelinating disease, idiopathic demyelinating polyneuropathy, Guillain-Barre syndrome, chronic inflammatory demyelinating polyneuropathy, hepatobiliary disease, infectious or autoimmune chronic Active hepatitis, primary biliary cirrhosis, granulomatous hepatitis, sclerosing cholangitis, inflammatory bowel disease, gluten-sensitive bowel disease, Whipple disease, autoimmune-mediated or immune-mediated skin disease, bullous dermatosis, Erythema multiforme, allergic rhinitis, atopic dermatitis, food hypersensitivity, urticaria, lung immune disease, eosinophilic pneumonia, idiopathic pulmonary fibrosis, hypersensitivity pneumonitis, transplantation related disease, graft Rejection or graft-versus-host disease, psoriatic arthritis, psoriasis, dermatitis, polymyositis/dermatomyositis, toxic epidermal necrosis, systemic scleroderma and sclerosis, inflammatory bowel disease related reactions, Crohn's disease, Ulcerative colitis, respiratory distress syndrome, adult respiratory distress syndrome (ARDS), meningitis, encephalitis, uveitis, colitis, glomerulonephritis, allergic conditions, eczema, asthma, T cell infiltration and chronic inflammatory Symptom with reaction, atherosclerosis, autoimmune myocarditis, leukocyte adhesion disorder, allergic encephalomyelitis, cytokine-mediated and T-lymphocyte mediated immune response with acute and delayed hypersensitivity, tuberculosis, Sarcoidosis, granulomatosis including Wegener's granulomatosis, agranulocytosis, vasculitis (including ANCA), aplastic anemia, diamond blackfan anemia, autoimmune hemolytic anemia AIHA), including immune hemolytic anemia, pernicious anemia, erythroblastic fistula (PRCA), factor VIII deficiency, type A hemophilia, autoimmune neutropenia, pancytopenia, leukopenia, Diseases with leukocyte leakage, central nervous system (CNS) inflammatory diseases, multiple organ injury syndrome, myasthenia gravis, antigen-antibody complex-mediated diseases, anti-glomerular basement membrane antibody diseases, anti-phospholipid antibody syndrome, allergy Neuritis, Behcet's disease, Castleman syndrome, Goodpasture syndrome, Lambert Eaton myasthenia syndrome, Raynaud's syndrome, Sjogren's syndrome, Stevens-Johnson syndrome, bullous pemphigoid, pemphigus, autoimmune multiple Endocrine disease, Reiter disease, stiff man syndrome, giant cell arteritis, immune complex nephritis, IgA nephropathy, IgM polyneuropathy or Ig Testis and ovary, including mediated neuropathy, idiopathic thrombocytopenic purpura (ITP), thrombotic thrombocytopenic purpura (TTP), autoimmune thrombocytopenia, autoimmune orchitis and autoimmune ovitis Autoimmune diseases, primary hypothyroidism, autoimmune endocrine diseases including autoimmune thyroiditis, chronic thyroiditis (Hashimoto's disease thyroiditis), subacute thyroiditis, idiopathic hypothyroidism, Addison's disease, Graves disease, autoimmune multiglandular syndrome (or multiglandular endocrine disorder syndrome), Sheehan syndrome, autoimmune hepatitis, lymphoid interstitial pneumonia (HIV), obstructive bronchiolitis (non-transplantable)) Vs. NSIP, Guillain-Barre syndrome, macroangitis (including rheumatic polymyalgia and giant cell (takayasu) arteritis), medium vasculitis (including Kawasaki disease and nodular polyarteritis), ankylos Spondylitis, Berger's disease (IgA nephropathy), rapidly progressive glomerulonephritis, primary biliary cirrhosis, celiac disease (glutenous bowel disease), cryoglobulinemia, and amyotrophic lateral sclerosis (ALS) Is included.

In another example, the methods, compositions, kits and principles described herein in the cancer context, may be utilized to collect DNA from cells or tissues involved in an neurological or neuropsychiatric disease or condition. As used herein, the term “neurological or neuropsychiatric disease or condition” refers to any disease or condition that affects the nervous system. Examples of neurological or neuropsychiatric disorders or symptoms include, but are not limited to, head trauma, stroke, stroke, ischemic stroke, hemorrhagic stroke, subarachnoid hemorrhage, intracranial hemorrhage, transient ischemic stroke Vascular dementia, corticobasal ganglion degeneration, encephalitis, epilepsy, Landau-Krffner syndrome, hydrocephalus, pseudobrain tumor, thalamic disease, meningitis, myelitis, movement disorder, essential tremor, spinal cord Disease, syringomyelia, Alzheimer's disease (early onset), Alzheimer's disease (late onset), multiple infarct dementia, Pick disease, Huntington's disease, Parkinson's disease, Parkinson's syndrome, dementia, frontotemporal dementia, Corticobasal degeneration, multisystem atrophy, progressive supranuclear palsy, Lewy body disease, Creutzfeldt-Jakob disease, Dundee-Walker syndrome, Friedreich ataxia, Masha Joseph disease, migraine, schizophrenia, mood disorders and depression, Lewy body dementia (DLB), frontotemporal dementia (FTD), various forms of vascular dementia (VD), subcortical vessels Sexual dementia (Binswanger disease), autism, developmental delay, motor neuron disease, amyotrophic lateral sclerosis (ALS), nerve or brain damage, cerebral hypoxia, cerebral palsy (CP), memory impairment, Movement disorders, cerebral cortex basal ganglia degeneration, various forms of atrophy, stroke-related diseases, cerebrovascular disorders, post-irradiation encephalopathy with convulsions, vascular parkinsonism, thalamic cerebrovascular disorders, chronic inflammatory Demyelinating polyneuropathy, alcohol-related dementia, semantic dementia, ataxia, atypical parkinsonism, dystonia, progressive supranuclear palsy, essential tremor, mild cognitive impairment, amyotrophic lateral sclerosis, Multiple Keratosis, neuropathy, pick disease, congo red affinity vascular disorder, Creutzfeldt-Jakob disease, AIDS dementia complex, depression, anxiety disorder, phobia, bell palsy, epilepsy, encephalitis, neuromuscular disorder, neurooncology Diseases, brain tumors, neurovascular disorders, neuroimmunological disorders, neuro-otological disorders, neurotrauma including spinal cord injuries, pain including neuropathic pain, pediatric and neuropsychiatric disorders, sleep disorders, Tourette syndrome, Alzheimer's disease with cerebral cortex basal ganglia degeneration, alcohol-related dementia, semantic dementia, multiple infarct dementia, Alzheimer's disease with Lewy body dementia, Parkinson with Lewy body dementia And Alzheimer's disease and Parkinson's disease with Lewy body dementia, frontotemporal with chronic inflammatory demyelinating polyneuropathy Type dementia, attention deficit/hyperactivity disorder, schizophrenia, obsessive compulsive disorder, mental retardation, autism spectrum disorder, opsoclonus myoclonus syndrome (OMS) convulsions, articulation disorder, learning disorder (ie reading or calculation), Deficiency in language or language skills, attention deficit disorder, amyloid disease, prion disease, tauopathy, alpha synuclein disease, cocaine, nicotine, alcohol, food, ecstasy, chat, caffeine, opium, heroin, marijuana, amphetamine, methamphetamine Or addiction symptoms resulting from at least one of gambling, and Fabry disease.

In another example, the methods, compositions, kits and principles described herein in the cancer context, may be utilized to collect DNA from cells or tissues involved in a kidney-related disease or condition. As used herein, the term “kidney-related disease or condition” refers to any disease or condition that affects the kidney or kidney system. Examples of kidney related diseases include, but are not limited to, chronic kidney disease, primary kidney disease, non-diabetic kidney disease, glomerulonephritis, interstitial nephritis, diabetic kidney disease, diabetic nephropathy, thread Glomerulosclerosis, rapidly progressive glomerulonephritis, renal fibrosis, Alport syndrome, insulin-dependent diabetic (IDDM) nephritis, mesangial proliferative glomerulonephritis, membranoproliferative glomerulonephritis, crescent-forming glomerulonephritis, Interstitial fibrosis, focal segmental glomerulosclerosis, membranous nephropathy, minimal change nephrotic syndrome, pauci-immune type rapidly progressive glomerulonephritis, IgA nephropathy, polycystic kidney disease, Dent's disease Nephrocytosis, Heyman nephritis, autosomal dominant (adult) polycystic kidney disease, autosomal recessive (pediatric) polycystic kidney disease, acute kidney injury, nephrotic syndrome, Ischemia, podocyte disease or disorder, proteinuria, glomerular disease, membranous glomerulonephritis, focal segmental glomerulonephritis, preeclampsia, eclampsia, kidney lesions, collagen vascular disease, benign orthostatic (postural)) Proteinuria, IgM nephropathy, membranous nephropathy, sarcoidosis, diabetes, drug-induced kidney damage, Fabry disease, amino acid urine, Fanconi syndrome, hypertensive nephrosclerosis, interstitial nephritis, sickle cell disease, Hemoglobinuria, myoglobinuria, Wegener's granulomatosis, type 1 glycogen storage disorder, chronic kidney disease, chronic renal failure, low glomerular filtration rate (GFR), renovascular sclerosis, lupus nephritis, ANCA positive pauci-immunonecrotic glomerulonephritis, chronic allograft nephropathy, nephrotoxicity, nephrotoxicity, renal necrosis, kidney damage, glomerular and tubular injury, renal dysfunction, nephritic syndrome, acute renal failure, chronic Renal failure, proximal tubule dysfunction, acute kidney transplant rejection, chronic kidney transplant rejection, non-IgA mesangial proliferative glomerulonephritis, post-infection glomerulonephritis, vasculitis with any type of nephropathy, any Hereditary kidney disease, any interstitial nephritis, kidney transplant failure, kidney cancer, kidney disease with other symptoms (eg, hypertension, diabetes, and autoimmune disease), Dent's disease, renal cystinosis, Hayman nephritis, primary kidney disease, collapsing glomerulopathy, dense deposit disease, cryoglobulinemia-related glomerulonephritis, Henoch-Schinlein disease, post-infection glomerulonephritis, bacterial endocarditis, microscopic occurrence Vasculitis, Churg-Strauss syndrome, anti-GBM antibody-mediated glomerulonephritis, amyloidosis, monoclonal immunoglobulin deposition, fibrillar thread Globe nephritis, immunotactoid glomerulopathy, ischemic tubular injury, drug-induced tubular interstitial nephritis, toxic tubular interstitial nephritis, infectious tubular interstitial nephritis, bacterial pyelonephritis, poly Virus-infected tubulointerstitial nephritis, metabolism-induced tubulointerstitial disease, mixed connective tissue disease, columnar nephropathy, uric acid crystals or oxalate crystals or drug-induced crystals resulting from infection with oma virus or HIV Crystalline nephropathy that can result from the deposition of tumors, acute cellular tubulointerstitial allograft rejection, neoplastic invasive disease due to lymphoma or post-transplant lymphoproliferative disease, obstructive kidney disease, vascular disease, Thrombotic microangiopathy, renovascular sclerosis, atheroembolic disease, mixed connective tissue disease, polyarteritis nodosa, calcineurin inhibitor-induced vascular disease, acute cellular vascular allograft rejection, acute humoral allograft Graft rejection, early renal dysfunction (ERFD), end stage renal disease (ESRD), renal vein thrombosis, acute tubular necrosis, acute interstitial nephritis, existing chronic kidney disease, renal artery stenosis, ischemic Includes nephropathy, uremia, drug-induced and toxic-induced chronic tubulointerstitial nephritis, reflux nephropathy, kidney stones, Goodpasture's syndrome, and hydronephrosis.

The methods, compositions, kits and principles described herein in the lung cancer context, may be utilized to collect DNA from cells or tissues involved in any cancer. As used herein, the term “cancer” refers to various types of malignant neoplasms, most of which can invade surrounding tissues and may metastasize to various sites (e.g., all in their entirety). The terms “neoplasm” and “tumor” refer to abnormal tissue that grows faster than normal by cell proliferation and continues to grow after the stimulus that initiated proliferation is removed (ibid). Such abnormal tissues are partially or completely lost in structural organization and functional coordination with normal tissues, which may be benign or malignant. Examples of general classifications of cancer include, but are not limited to, carcinomas (i.e., malignant tumors derived from epithelial cells such as common breast cancer, prostate cancer, lung cancer, and colon cancer), sarcomas (i.e., malignant tumors derived from connective tissue or mesenchymal cells), lymphomas (i.e., malignant lesions derived from hematopoietic cells), leukemias (i.e., malignant lesions derived from hematopoietic cells), germ cell tumors (i.e., tumors derived from totipotent cells). In adults, it is most common in the testis or ovary, in fetuses, infants, and young children, it is most common in the body normal, especially at the tip of the coccyx, and blastic tumors (i.e., immature or embryonic tissue and tumors that are usually malignant). Examples of neoplastic types that are intended to be encompassed by the present invention include, but are not limited to, neural tissue cancer, hematopoietic tissue cancer, breast cancer, skin cancer, bone cancer, prostate cancer, ovarian cancer, Uterine cancer, cervical cancer, liver cancer, lung cancer, brain cancer, laryngeal cancer, gallbladder cancer, pancreatic cancer, rectal cancer, parathyroid cancer, thyroid cancer, adrenal cancer, cancer of immune system, head and neck cancer, colon cancer, stomach cancer, Neoplasms associated with bronchial cancer, and/or kidney cancer.

### Techniques for Isolating Macrophages

In some embodiments, the methods of the present disclosure may relate to techniques for isolating phagocytes. In some embodiments, phagocytes are isolated from a biological sample or fluid collected from a subject. In some embodiments, the phagocyte to be isolated is a macrophage.

Approaches for isolating macrophages are well known by those familiar with the art, as a variety of methods and kits are publicly available, often as commercial products. Examples of techniques for the isolation of macrophages from various biological fluids are described for instance in Rios et al. “Isolation and Differentiation of Human Macrophages” Methods Mol Biol. 2017; 1527:311-320; Bølling et al. “Isolating and culturing of sputum macrophages: A potential ex vivo/in vitro model” Exp Lung Res. 2018 August; 44(6):312-322; and Sikkeland et al. “Macrophage enrichment from induced sputum” Thorax. 2007 June; 62(6):558-9, each of which are herein incorporated by reference. Briefly, macrophages may be isolated by means of density gradient centrifugation (e.g., Percoll gradient separation) and/or immunomagnetic bead separation in order to achieve a high degree of macrophage purity.

### Techniques for Separating Macrophage Lysosomes

In some embodiments, the methods of the present disclosure may relate to techniques for isolating various membrane-enclosed vesicles and/or organelles from phagocytes, e.g., vesicles, vacuoles, and/or organelles that may contain tumor-derived DNA. In some embodiments, lysosomes are isolated from phagocytes. In some embodiments, lysosomes are isolated from macrophages.

Approaches for isolating lysosomes from cells, including isolation of lysosomes from macrophages specifically, are well known to those familiar with the art as a variety of methods and kits are publicly available, including commercial products. Techniques for the isolation of macrophage lysosomes are described, for instance, in Rofe and Pryor “Purification of Lysosomes Using Supraparamagnetic Iron Oxide Nanoparticles (SPIONs)” Cold Spring Harb Protoc. 2016, which is incorporated by reference herein. Briefly, in one embodiment, macrophage lysosomes may be isolated by allowing macrophages to uptake magnetic particles, disrupting cell membranes, and magnetically separating lysosomes. Lysosomes may also be isolated by ultracentrifugation as described, for example, in Aguado et al. “Isolation of Lysosomes from Mammalian Tissues and Cultured Cells” Proteostasis. 2016, Methods in Molecular Biology, vol 1449, which is incorporated by reference herein.

Other vesicles, vacuoles, and/or organelles may also be isolated from phagocytes, e.g. macrophages, including but not limited to phagosomes, autophagosomes, and endosomes. Without wishing to be bound by theory, phagosomes potentially contain intact tumor cells that have been phagocytosed, prior to fusion with lysosomes for degradation.

Autophagosomes and endosomes may each also contain tumor-derived DNA in the form of cell free DNA (cfDNA) that has been endocytosed from the extracellular environment (e.g. blood, airway surface liquid, etc.). Techniques for the isolation of lysosomes are generally also applicable to the isolation of phagosomes, as is well known in the art, and includes isolation by magnetic separation or ultracentrifugation (see, e.g., Steinhauseret et al. “Immunomagnetic Isolation of Pathogen-Containing Phagosomes and Apoptotic Blebs from Primary Phagocytes”. Current Protocols in Immunology. 2014, 105: 14.36.1-14.36.26; and Hartlova et al. “Isolation and Western Blotting of Latex-Bead Phagosomes to Track Phagosome Maturation” Phagocytosis and Phagosomes. 2017, Methods in Molecular Biology, vol 1519, which is incorporated by reference herein). Autophagosomes and endosomes may be isolated similarly by means of magnetic separation or ultracentrifugation, as is well known in the art (see, e.g., Takahashi et al. “Magnetic Separation of Autophagosomes from Mammalian Cells Using Magnetic-Plasmonic Hybrid Nanobeads”. ACS Omega. 2017, 2(8), 4929-4937; Takahashi and Maenosono “Magnetic Separation of Organelles using Magnetic Beads”. IOP Publishing 2018, Magnetic Nanoparticles for Medical Diagnostics, 2:1-18; and de Araújo et al. “Isolation of Early and Late Endosomes by Density Gradient Centrifugation”. Cold Spring Harb Protoc. 2015 Nov. 2; 2015(11):1013-6, which are incorporated by reference herein). Various kits for the purification of phagosomes, autophagosomes, and endosomes are also widely commercially available and are well known in the art.

### DNA Extraction Methods

Any suitable method known to one of ordinary skill in the art may be used to extract the DNA, including the tumor-derived DNA, from the phagocytic macrophages. The extraction of nucleic acid from cells, such as phagocytic macrophages, can involve cell lysis, inactivation of cellular nucleases and/or separation steps of the desired nucleic acid material from cellular debris. Common lysis procedures can include, but are not limited to, mechanical disruption (e.g., grinding, hypotonic lysis), chemical treatment (e.g., detergent lysis, chaotropic agents, thiol reduction), and enzymatic digestion (e.g., proteinase K). In the present invention, the biological sample may be first lysed in the presence of a lysis buffer, chaotropic agent (e.g., salt) and proteinase or protease. Cell membrane disruption and inactivation of intracellular nucleases may be combined. For instance, a single solution may contain detergents to solubilize cell membranes and strong chaotropic salts to inactivate intracellular enzymes. After cell lysis and nuclease inactivation, cellular debris may easily be removed by filtration or precipitation.

In another example, the methods of the present invention may be used in conjunction with any known technique suitable for the extraction, isolation or purification of nucleic acids, including, but not limited to, cesium chloride gradients, gradients, sucrose gradients, glucose gradients, centrifugation protocols, boiling, Microcon 100 filter, Chemagen viral DNA/RNA 1 k kit, Chemagen blood kit, Qiagen purification systems, Qiagen MinElute kits, QIA DNA blood purification kit, HiSpeed Plasmid Maxi Kit, QIAfilter plasmid kit, Promega DNA purification systems, MangeSil Paramagnetic Particle based systems, Wizard SV technology, Wizard Genomic DNA purification kit, Amersham purification systems, GFX Genomic Blood DNA purification kit, Invitrogen Life Technologies Purification Systems, CONCERT purification system, Mo Bio Laboratories purification systems, UltraClean BloodSpin Kits, and UlraClean Blood DNA Kit.

The kits, compositions, and methods contemplate any suitable DNA extraction method known in the art. Suitable examples are described in: Eslami et al., “Comparison of Three Different DNA Extraction Methods for Linguatula Serrata as a Food-Borne Pathogen,” Iran J Parasitol. 2017; 12(2):236-242; Dilhari et al. “Evaluation of the impact of six different DNA extraction methods for the representation of the microbial community associated with human chronic wound infections using a gel-based DNA profiling method.” AMB Expr (2017) 7:179. DOI 10.1186/s13568-017-0477-z; Kelly M. Elkins. “Chapter 4: DNA extraction.” Forensic DNA biology (2013): 39-52; M. Dairawan and P. Shetty, “The Evolution of DNA Extraction Methods,” American Journal of Biomedical Science & Research, Mar. 11, 2020, Vol. 8(1), p. 39-45; each of which are incorporated by reference in their entireties.

### DNA Sequencing Methods

In various embodiments, assaying of tumor-derived DNA may involve obtaining the sequence of the tumor-derived DNA or a portion thereof. This can include obtaining the sequence of an amplified product of the tumor-derived DNA.

In some embodiments, the isolated tumor-derived DNA can be sequenced by next generation sequencing methods. In some embodiments, the next generation sequencing method comprises a method selected from the group consisting of Ion Torrent, Illumina, SOLiD, 454; Massively Parallel Signature Sequencing, solid phase reversible dye terminator sequencing; and DNA nanoball sequencing. As used herein, “next generation sequencing” refers to the speeds that were not possible with conventional sequencing methods (e.g., Sanger sequencing) by reading thousands of millions of sequencing reactions simultaneously.

Next generation sequencing techniques and sequencing primer designs are well known in the art (e.g., Shendure, et al., “Next-generation DNA sequencing,” Nature, 2008, vol. 26, No. 10, 1135-1145; Mardis, “The impact of next-generation sequencing technology on genetics, “Trends in Genetics, 2007, vol. 24, No. 3, pp 133-141; Su, et al., “Next-generation sequencing and its applications in molecular diagnostics,” Expert Rev Mol Diagn, 2011, 11 (3): 333-43; Zhang et al., “The impact of next-generation sequencing on genomics,” J Genet Genomics, 2011, 38 (3): 95-109; (Nyren, P. et al. Anal Biochem 208: 17175 (1993): Bentley, DR Curr Opin Genet Dev 16: 545-52 (2006); Strausberg, R L, et al. Drug Disc Today 13: 569-77 (2008); U.S. Pat. Nos. 7,282,337; 7,279,563; 7,226,720; 7,220,549; 7,169,560; see and No. 20070070349); U.S. Pat. Nos. 6,818,395; 6,911,345; U.S. Patent Application Publication No. 2006/0252077; No. 2007/0070349. The entire contents of these general references on next-generation sequencing are incorporated herein by reference.

### Techniques for Direct Sequencing of Tumor DNA in Macrophages

In some embodiments, the methods of the present disclosure may relate to sequencing of tumor-derived DNA from collected phagocytes (e.g., macrophages). In some embodiments, the methods contemplated herein may relate to sequencing of tumor-derived DNA from individual phagocytes (e.g., macrophages), i.e., by means of single-cell sequencing technologies. Single-cell sequencing refers to any sequencing technique by which DNA from individual cells is sequenced using NGS in such a way that the sequences that are obtained may be traced to a individual isolated cell and distinguished from sequences obtained from other cells. In this way, sequencing of all phagocytosed DNA in a single phagocyte may be used to determine the genome of a single phagocytosed tumor cell.

A wide variety of approaches for single-cell sequencing are known in the art. Common techniques for single-cell sequencing include multiple displacement amplification (MDA), whole genome amplification (WGA), multiple annealing and looping-based amplification cycles (MALBAC), as well as derivative techniques thereof. Techniques for single-cell DNA sequencing are described, for example, in Spits et al. “Whole-genome multiple displacement amplification from single cells” Nat Protoc (2006) 1, 1965-1970; Zong et al. “Genome-wide detection of single-nucleotide and copy-number variations of a single human cell” Science (2012) 338(6114):1622-6; and Huang et al. “Single-Cell Whole-Genome Amplification and Sequencing: Methodology and Applications”. Annu Rev Genomics Hum Genet. (2015) 16:79-102, each of which are incorporated by reference herein.

### Techniques for Analyzing DNA and Cancer Biomarkers

In various embodiments, analysis of tumor-derived DNA involves sequencing the DNA by any means known in the art and assessing the sequenced DNA for one or more genetic markers indicative of a disease or condition, such as cancer.

In some embodiments, analysis of sequenced DNA involves analyzing the DNA for the presence of mutations that may be or are known to be positively correlated with one or more types of cancer. Analysis may include, for instance, analysis of mutations with specific genes, referred to as cancer biomarkers, which are known to be oncogenic. Mutations may be missense mutations, where a single nucleotide base is changed, as well as insertions and deletions of nucleotide bases. Mutations may increase or decrease the expression of genes in which they occur or modify the function of proteins or miRNAs expressed from these genes. Cancer biomarkers may include genes that encode proteins or micro RNA (miRNA). Protein and miRNA cancer biomarkers are widely known in the art, as are specific mutations which contribute to the development of cancer. Examples of cancer biomarkers which when mutated can contribute to various types of cancer, such as lung cancer, are described in U.S. Pat. Nos. 9,249,465, 9,291,625, 10,338,075, and 10,620,228, which are incorporated by reference herein.

In some embodiments, analysis of sequenced DNA involves analyzing the DNA for copy number changes. Copy number changes occur when tumor cells duplicate or delete all or part of their genome, resulting in an increase or decrease in the number of genes that may be detected by sequencing, compared to non-cancerous cells. Copy number changes in cancer biomarkers are also known to contribute to the development of cancer by increasing or decreasing expression of the biomarker.

In some embodiments, analysis of sequenced DNA involves further analyzing the DNA for epigenetic changes, such those of the DNA methylation profile, changes in which are also widely known to modify genetic expression.

In some embodiments, analysis of sequenced DNA involves assessing the tumor fraction of a sample, defined as the proportion of sequenced DNA that is likely to have originated from a tumor cell. Tumor fraction may be determined for the phagocytosed DNA present in a single phagocyte (i.e., when conducting single-cell sequencing of phagocytes) or in a population of phagocytes, such as, for instance, all or a subset of phagocytes in a collected biological sample or fluid.

DNA analysis may involve an amplification step. Nucleic acid amplification methods include, without limitation, polymerase chain reaction (PCR) (U.S. Pat. No. 5,219,727) and its variants such as in situ polymerase chain reaction (U.S. Pat. No. 5,538,871), quantitative polymerase chain reaction (U.S. Pat. No. 5,219,727), nested polymerase chain reaction (U.S. Pat. No. 5,556,773), self-sustained sequence replication and its variants, transcriptional amplification system and its variants, Qb Replicase and its variants, cold-PCR, or any other nucleic acid amplification methods, followed by the detection of the amplified molecules using techniques well known to those of skill in the art. Especially useful are those detection schemes designed for the detection of nucleic acid molecules if such molecules are present in very low numbers. The foregoing references are incorporated herein for their teachings of these methods.

DNA analysis may involve detection of one or more genetic aberration, which can include, without limitation, over-expression of a gene (e.g., oncogenes) or a panel of genes, under-expression of a gene (e.g., tumor suppressor genes such as p53 or RB) or a panel of genes, alternative production of splice variants of a gene or a panel of genes, gene copy number variants (CNV) (e.g. DNA double minutes), nucleic acid modifications (e.g., methylation, acetylation and phosphorylations), single nucleotide polymorphisms (SNPs), chromosomal rearrangements (e.g., inversions, deletions and duplications), and mutations (insertions, deletions, duplications, missense, nonsense, synonymous or any other nucleotide changes) of a gene or a panel of genes, which mutations, in many cases, ultimately affect the activity and function of the gene products. Methylation profiles may be determined by Illumina DNA Methylation OMA003 Cancer Panel. SNPs and mutations can be detected by hybridization with allele-specific probes, enzymatic mutation detection, chemical cleavage of mismatched heteroduplex, ribonuclease cleavage of mismatched bases, mass spectrometry (U.S. Pat. Nos. 6,994,960, 7,074,563, and 7,198,893), nucleic acid sequencing, single strand conformation polymorphism (SSCP), denaturing gradient gel electrophoresis (DGGE), temperature gradient gel electrophoresis (TGGE), restriction fragment length polymorphisms (RFLP), oligonucleotide ligation assay (OLA), allele-specific PCR (ASPCR) (U.S. Pat. No. 5,639,611), ligation chain reaction (LCR) and its variants, flow-cytometric heteroduplex analysis (WO/2006/113590) and combinations/modifications thereof Notably, gene expression levels may be determined by the serial analysis of gene expression (SAGE) technique. In general, the methods for analyzing genetic aberrations are reported in numerous publications, not limited to those cited herein, and are available to skilled practitioners. The appropriate method of analysis will depend upon the specific goals of the analysis, the condition/history of the patient, and the specific cancer(s), diseases or other medical conditions to be detected, monitored or treated. The forgoing references are incorporated herein for their teachings of these methods.

It should be understood that this invention is not limited to the particular methodologies, protocols and reagents, described herein, which may vary. The terminology used herein is for the purpose of describing particular embodiments only, and is not intended to limit the scope of the present invention, which is defined solely by the claims.

### Methods for Use

In another aspect, the present disclosure relates to various methods for increasing the concentration of tumor-derived DNA in one or more phagocytic cell types of a subject. In some embodiments, these methods comprise administering to a subject an effective amount of one or more agents capable of increasing the concentration of tumor-derived DNA in a phagocyte, e.g. a macrophage. In some embodiments, these methods comprise administering to a subject an effective amount of one or more agents capable of increasing the concentration of tumor-derived DNA in lysosomes of a phagocyte, e.g. a macrophage. In some embodiments, these methods comprise administering to a subject an effective amount of one or more agents that inhibit deoxyribonucleases, one or more agents for modifying the microenvironment of phagocytes (e.g., the lysosomal microenvironment), and/or one or more agents for enhancing recovery of phagocytes (e.g., by impairing adherence of phagocytes).

In some embodiments, these methods include a step of collecting phagocytes (e.g., macrophages) or a biological sample or fluid containing phagocytes (e.g., blood, sputum, etc.) from a subject after administering one or more agents for increasing the concentration of tumor-derived DNA in phagocytes. In some embodiments, a biological sample or fluid containing phagocytes is collected from the subject at least 5 minutes, at least 10 minutes, at least 15 minutes, at least 30 minutes, at least 1 hour, at least 2 hours, at least 3 hours, at least 4 hours, at least 5 hours, at least 10 hours, or at least 20 hours, but not longer than 25 hours, after administering the one or more agents for increasing the concentration of tumor-derived DNA in phagocytes. In some embodiments, these methods further include isolating phagocytes (e.g., macrophages) from the collected biological sample or fluid. In some embodiments, these methods further include isolating lysosomes from phagocytes (e.g., macrophages). In some embodiments, these methods further include isolating tumor-derived DNA from the collected biological sample or fluid (e.g., blood, sputum, etc.) or isolated phagocytes (e.g., macrophages).

In some embodiments, these methods include a step of detecting and/or analyzing tumor-derived DNA after collecting a biological sample or fluid containing phagocytes, isolating phagocytes, isolating lysosomes from phagocytes, and/or isolating tumor-derived DNA. Tumor-derived DNA may be detected by any means known in the art for the detection of DNA species, including, but not limited to, polymerase chain reaction (PCR), DNA gel electrophoresis, DNA precipitation, DNA spectroscopy (e.g., UV/Vis), DNA precipitation, DNA digestion, Southern blotting, hybridization with fluorescent or radiolabeled oligonucleotide probes, or DNA sequencing. In some embodiments, analysis of tumor-derived DNA may be conducted by way of sequencing the tumor-derived DNA through any means known in the art (e.g., fragment analysis, Sanger sequencing, capillary electrophoresis, single-cell sequencing, next generation sequencing (NGS), etc.) and analyzing the sequenced DNA for indicators that it originated from a tumor cell. Analysis of sequenced DNA may include analysis of genetic markers indicative of cancer, such as the presence of mutations known to be positively correlated with cancer or copy number alterations. Analysis of sequenced DNA may also include an analysis of the proportion of all phagocytosed DNA sequenced from a sample that is likely to have originated from a tumor cell (i.e., tumor fraction). In some embodiments, this analysis may be subsequently used to identify a subject as having cancer, to determine the type of cancer a subject has, or to predict the progression of cancer in a subject. In some embodiments, a subject identified as having cancer may be administered one or more treatments for cancer, as are widely known in the art.

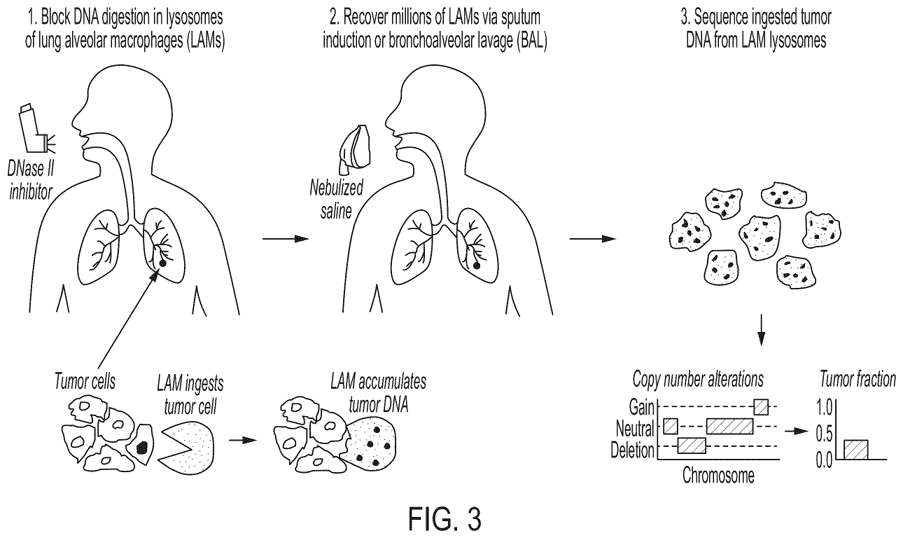

In some embodiments, these methods specifically relate to administering to a subject an effective amount of one or more agents capable of increasing the concentration of tumor-derived DNA in a phagocyte, e.g. a macrophage, located in the subject's lungs (FIG. 3). In some embodiments, these methods include a step of collecting macrophages (e.g., alveolar macrophages) or a biological sample or fluid containing macrophages (e.g., sputum) from a subject after administering one or more agents for increasing the concentration of tumor-derived DNA in macrophages of the subject's lungs. In some embodiments, a biological sample or fluid containing macrophages from a subject's lungs is collected by way of sputum induction or bronchoalveolar lavage (BAL). In some embodiments, these methods further include isolating macrophages from the biological sample or fluid collected from the lungs. In some embodiments, these methods further include isolating lysosomes from macrophages collected from the lungs. In some embodiments, these methods further include isolating tumor-derived DNA from the collected biological sample or fluid or macrophages from the lungs. In some embodiments, these methods further involve detection and/or analysis of the tumor-derived DNA and may further involve sequencing of the DNA and analysis of the sequenced DNA for indications of cancer. In some embodiments, these methods may be used to identify a subject as having lung cancer. In some embodiments, these methods further comprise administering to a subject one or more treatments for lung cancer.

In some embodiments, the subject is a human patient. In some embodiments, the subject is known to have, is suspected of having, or is at risk for cancer, such as, but not limited to, lung cancer, colorectal cancer, breast cancer, pancreatic cancer, prostate cancer, bladder cancer, kidney cancer, thyroid cancer, uterine cancer, cervical cancer, ovarian cancer, testicular cancer, esophageal cancer, stomach cancer, liver cancer, brain cancer, peritoneal cancer, lymphoma, leukemia, multiple myeloma, neuroblastoma, osteosarcoma, and soft tissue sarcoma.

### Pharmaceutical Compositions

In another aspect, the present disclosure relates to pharmaceutical compositions for increasing the concentration of tumor-derived DNA in one or more phagocytic cell types of a subject. In some embodiments, the contemplated pharmaceutical compositions comprise one or more agents capable of increasing the concentration of tumor-derived DNA in a phagocyte, e.g. a macrophage. In some embodiments, the contemplated pharmaceutical compositions comprise one or more agents capable of increasing the concentration of tumor-derived DNA in lysosomes of a phagocyte, e.g. a macrophage. In some embodiments, the contemplated pharmaceutical compositions comprise one or more agents that inhibit deoxyribonucleases, one or more agents for modifying the microenvironment of phagocytes (e.g., the lysosomal microenvironment), and/or one or more agents for enhancing recovery of phagocytes (e.g., by impairing adherence of phagocytes).

The contemplated pharmaceutical compositions may be in any form suitable for administration to a subject, e.g., a liquid composition, a solid composition, a gel composition, or aerosolized compositions thereof. Other compositions are also contemplated, including those that may delivered by transdermal patches, emulsions, foams, granules, implants, pellets, pills, sprays, suppositories, suspensions, tablets, and the like, so long as agent(s) may be delivered and increase the concentration of phagocytosed DNA in phagocytes of one or more biological tissues or liquids. Such compositions will generally comprise a carrier of some sort, for example a solid carrier or a liquid carrier such as water, petroleum, animal or vegetable oils, mineral oil, or synthetic oil. Physiological saline solution, or glycols such as ethylene glycol, propylene glycol or polyethylene glycol may also be included. Such compositions and preparations generally contain at least 0.1 wt % of the active agent. Such formulations are well known in the art.

In some embodiments, the contemplated pharmaceutical compositions may comprise one or more of a pharmaceutically acceptable excipients, carriers, buffers, stabilizers, delivery agents, isotonicizing agents, preservatives or antioxidants, or other materials well known to those skilled in the art, in addition to one or more agents for increasing the concentration of tumor-derived DNA in a phagocyte, e.g. a macrophage. Such materials should be non-toxic and should not interfere with the efficacy of the active ingredient. The precise nature of the excipients, carriers, buffers, stabilizers, delivery agents, isotonicizing agents, preservatives or antioxidants, or other materials may depend on the route of administration, e.g., intravenously, orally, or through inhalation.

For administration through inhalation, the contemplated pharmaceutical composition will be in the form of a liquid or dry powder that is capable of being aerosolized, e.g. by an inhalation device for oral and/or nasal inhalation for delivery to the lungs. In embodiments where the pharmaceutical composition is a liquid that can be aerosolized, it will be in the form of an acceptable aqueous solution which is pyrogen-free and has suitable pH, isotonicity, and stability.

The contemplated pharmaceutical composition may contain microspheres, especially in embodiments where the pharmaceutical composition is in the form of a dry powder that can be aerosolized. In embodiments where the pharmaceutical composition contains microspheres, the microspheres may be physically associated with the agent(s) capable of increasing the concentration of tumor-derived DNA in one or more phagocytic cell types. In some embodiments, the microspheres are produced by spray drying a liquid feed stock comprising the agent(s) and a material suitable for forming the structural matrix of a microsphere. The structural matrix of a microsphere may be composed of a phospholipids, such as dipalmitoylphosphatidylcholine, disteroylphosphatidylcholine, diarachidoylphosphatidylcholine dibehenoylphosphatidylcholine, short-chain phosphatidylcholines, long-chain saturated phosphatidylethanolamines, long-chain saturated phosphatidylserines, long-chain saturated phosphatidylglycerols, long-chain saturated phosphatidylinositols, glycolipids, ganglioside GM1, sphingomyelin, phosphatidic acid, cardiolipin; lipids bearing polymer chains such as polyethylene glycol, chitin, hyaluronic acid, or polyvinylpyrrolidone; lipids bearing sulfonated mono-, di-, and polysaccharides; fatty acids such as palmitic acid, stearic acid, and oleic acid; cholesterol, cholesterol esters, and cholesterol hemisuccinate. In some embodiments, the microspheres are porous or perforated. In some embodiments, the microspheres are hollow, encompassing a relatively large void at their center. In some embodiments, the microspheres are approximately spherical with an average diameter of about 0.5 μm to about 100 μm across. In some embodiments, the microspheres enhance delivery or uptake of one or more bound agents by cells of the lungs (e.g. alveolar macrophages), compared to pharmacological compositions that do not contain microspheres. Methods for producing and administering pharmaceutical compositions containing microspheres bound to bioactive agents are well known in the art, such as in U.S. Pat. No. 9,554,993, which is incorporated by reference herein.

In some embodiments, pharmaceutical compositions for inhaled administration may contain a propellant. Pharmaceutically acceptable propellants having low boiling points, low vapor pressures, and no toxicity are well known in the art. Pharmaceutically acceptable propellants include chlorofluorocarbon (CFC) propellants as well as hydrofluoroalkane (HFA) propellants, the latter of which are generally preferred. Several examples of CFC and HFA propellants that may be used to administer inhaled pharmaceutical compositions, particularly through the use of an inhaler device, are described in Myrdal et al. “Advances in metered dose inhaler technology: formulation development.” AAPS PharmSciTech vol. 15,2 (2014): 434-55, which is incorporated by reference herein.

For intravenous, cutaneous, or subcutaneous injection, the contemplated pharmaceutical composition will be in the form of a parenterally acceptable aqueous solution which is pyrogen-free and has suitable pH, isotonicity, and stability. Those of relevant skill in the art are knowledgeable in means of preparing suitable solutions using, for example, solutions of containing the active agent(s) in, e.g., physiological saline, a dispersion prepared with glycerol, liquid polyethylene glycol, or oils.

In embodiments where the contemplated pharmaceutical composition is a liquid, the composition may be formulated to have a pH between about 3.0 and 9.0, or preferably between about 4.5 and 8.5. Ideally, a liquid pharmaceutical composition has a pH between about 5.0 and 8.0. The pH of a composition can be maintained by the use of a buffer such as acetate, citrate, phosphate, succinate, Tris or histidine, typically employed in the range from about 1 mM to 50 mM. The pH of compositions can otherwise be adjusted by using physiologically acceptable acids or bases.

The pharmaceutical compositions contemplated herein may also comprise preservatives. Preservatives are generally included in pharmaceutical compositions to retard microbial growth, thereby extending the shelf life of the compositions and allowing multiple use packaging. Examples of preservatives include phenol, meta-cresol, benzyl alcohol, parahydroxybenzoic acid and its esters, methyl paraben, propyl paraben, benzalconium chloride and benzethonium chloride. Preservatives are typically employed in the range of about 0.1 to 1.0% (w/v).

Compositions containing one or more agents for increasing the concentration of tumor-derived DNA in phagocytes are preferably administered to a subject in a sufficiently effective amount (i.e., for achieving an increase the concentration of phagocytosed DNA within phagocytes of the subject). Examples of the techniques and protocols relevant for establishing the effective amount of a pharmaceutical composition can be found in Handbook of Pharmaceutical Additives, 2nd Edition (eds. M. Ash and I. Ash), 2001 (Synapse Information Resources, Inc., Endicott, New York, USA); Remington's Pharmaceutical Sciences, 20th Edition, 2000, pub. Lippincott, Williams & Wilkins; and Handbook of Pharmaceutical Excipients, 2nd edition, 1994).

### Kits for Increasing the Concentration of Phagocytic Tumor DNA

In another aspect, the present disclosure relates to kits for increasing the concentration of tumor-derived DNA in one or more phagocytic cell types of a subject, wherein such a kit comprises sufficient agent(s) for increasing the concentration of tumor-derived DNA in phagocytes of a subject, as well as instructions for administration of the kit. Such a kit may be utilized in the course of determining whether a subject has a disease associated with phagocytosed DNA. In particular instances, such a kit may be utilized in the course of determining whether a human subject possesses mutations in phagocytosed DNA that are indicative of cancer.

In some embodiments, the kits comprise one or more of a pharmaceutically acceptable excipients, carriers, buffers, stabilizers, delivery agents, isotonicizing agents, preservatives or antioxidants, or other materials well known to those skilled in the art. Such materials should be non-toxic and should not interfere with the efficacy of agent(s) for increasing the concentration of tumor-derived DNA in phagocytes. The precise nature of the excipients, carriers, buffers, stabilizers, delivery agents, isotonicizing agents, preservatives or antioxidants, or other materials may depend on the route of administration, e.g., intravenously, orally, or through inhalation.

In some embodiments, the kits are to be stored below 50° C., below 40° C., below 30° C., below 20° C., below 10° C., below 0° C., below −10° C., or below −20° C. such that the one or more agents for increasing the concentration of tumor-derived DNA are relatively stable over time.

In some embodiments, the agent(s) of the kit and any pharmacologically acceptable excipients, carriers, buffers, stabilizers, delivery agents, isotonicizing agents, preservatives or antioxidants with which they are stored are contained within a container that maintains the sterility and stability of the agent(s) prior to administration. In some embodiments, the container is also device that may be used to administer the agent(s) and any excipients, etc., to a subject. In some embodiments, the agents are stored in a glass vial or equivalent container from which the agent(s) may be delivered to a subject intravenously by means of a syringe fitted with a hypodermic needle. In some embodiments, the agents are stored in an inhalation device, such as a meter-dosed inhaler, a soft mist inhaler, a dry powder inhaler, or a nebulizer, from which the agent(s) may be delivered to the subject by inhalation of the pharmaceutical composition it contains.

In some embodiments, the kits are further utilized for the collection of a phagocyte-containing biological sample from the subject after the kit is used to increase the concentration of phagocytic DNA in a subject. In embodiments where a kit is utilized for the collection of a phagocyte-containing biological sample, the kit may contain devices, containers, and/or solutions for use in the recovery of one or more biological samples. In some embodiments, collection of a biological sample is conducted at least 5 minutes, at least 10 minutes, at least 15 minutes, at least 30 minutes, at least 1 hour, at least 2 hours, at least 3 hours, at least 4 hours, at least 5 hours, at least 10 hours, or at least 20 hours, but not longer than 25 hours, after using the kit to increase the concentration of phagocytosed DNA in phagocytic cells of the subject.

In some embodiments, the kits are in the format of liquid biopsy kits. In embodiments where a kit is utilized as a liquid biopsy kit, the kit can be used to increase the concentration of phagocytosed DNA in one or more phagocytic cell types of a subject and then optionally to collect and/or analyze a phagocyte-containing biological fluid from the subject. In some embodiments, the kit is in the format of a blood biopsy kit, wherein use of the kit to boost levels phagocytosed DNA is followed by collection and analysis of blood from the subject. In some embodiments, the kit is in the format of a sputum biopsy kit, wherein use of the kit to boost levels phagocytosed DNA is followed by collection and analysis of sputum from the subject, optionally after sputum induction in the subject. In some embodiments, the kit is in the format of a BAL fluid biopsy kit, wherein use of the kit to boost levels phagocytosed DNA is followed by BAL and subsequent collection and analysis of BAL fluid from the subject.

In some embodiments, analysis of phagocytosed DNA is further conducted directly in the collected biological sample or fluid (e.g., sputum, BAL fluid). In some embodiments, analysis of phagocytosed DNA is further conducted after isolating phagocytes (e.g., macrophages) from the collected biological sample or fluid. In some embodiments, analysis of phagocytosed DNA is further conducted after isolating lysosomes from isolated phagocytes (e.g., macrophages). In some embodiments, analysis of phagocytosed DNA is further conducted after isolating the phagocytosed DNA from the biological sample or fluid (e.g., sputum, BAL fluid) or from isolated phagocytes from the biological sample or fluid (e.g., macrophages). In some embodiments, analysis of phagocytosed DNA involves sequencing of phagocytosed DNA and identification of one or more disease-causing mutations present in the phagocytosed DNA. In some embodiments, the disease-causing mutations in question are associated with a cancer, such as lung cancer, colorectal cancer, breast cancer, pancreatic cancer, prostate cancer, bladder cancer, kidney cancer, thyroid cancer, uterine cancer, cervical cancer, ovarian cancer, testicular cancer, esophageal cancer, stomach cancer, liver cancer, brain cancer, peritoneal cancer, lymphoma, leukemia, multiple myeloma, neuroblastoma, osteosarcoma, or soft tissue sarcoma.

### EXAMPLES

### Example 1—Harnessing Macrophages for Collection and Detection of Tumor DNA

Lung cancer is the leading cause of cancer-related mortality, accounting for approximately 1.7 million deaths annually worldwide. Survival is strongly associated with stage at diagnosis (five-year overall survival: 56% in localized, 29% in regional, and 5% in distant-stage disease), and yet only a small minority of patients are diagnosed with localized or early-stage disease. Identification of lung cancer at early-stage disease, when surgical resection may be curative, could substantially improve these poor patient outcomes. As a result, there has been significant focus on early detection efforts. While the advent of lung cancer screening with annual low dose CT scans in high-risk patient populations and the general increased use of CT imaging in clinical practice have facilitated the identification of hundreds of thousands of pulmonary nodules at risk for cancer per year, one of the major challenges moving forward is determining which of these nodules are malignant and which are benign.

The current clinical standard for determining whether pulmonary nodules are malignant involves obtaining a pathologic diagnosis, typically using cytology or histologic assessment of tissue acquired during a biopsy. The diagnostic yields of cytology and biopsies vary depending on nodule size, accessibility, and procedural technique, and they are particularly low for small, peripheral nodules given the difficulties of physically targeting them successfully. For example, the sensitivities of bronchoalveolar lavage fluid (BALF) cytology and transbronchial needle aspiration (TBNA) of suspected peripheral based lung cancers <2 cm in diameter are 29% and 50-60% respectively. This limited sensitivity is particularly problematic as approximately 70% of lung cancers develop in peripheral lung fields. While there are more definitive approaches, such as surgical-based biopsies and resection, pursuing these options for most nodules is not practical given the risks of the procedure and the high proportion of benign lesions. Overall, the net limitations and risks of acquiring a pathologic diagnosis for most of these small, peripheral nodules outweigh benefits, and, as a result, these nodules are instead followed by repeat imaging over months to years to assess if they continue to grow or spread. This approach is inherently limited, as it is predicated on allowing potential malignant lesions to progress before arriving at a diagnosis.

There have been multiple proposed solutions to improve upon the accuracy of diagnostic testing while simultaneously minimizing the associated risks of specimen acquisition. These have included serum biomarkers, automated sputum analysis to identify tumor cells or tumor antigens, exhaled breath analysis of volatile organic compounds to identify tumor signatures, and nasal swab classifiers leveraging “field of injury” to assess differential gene expression. Unfortunately, many of the current iterations of these diagnostics either have limited accuracy, have limited scope, or require further testing, and as a result, have yet to be adopted for widespread clinical use.

More recently, liquid biopsies have garnered tremendous interest in filling this ongoing diagnostic need. Liquid biopsies involve measurement of tumor-derived cell free DNA (cfDNA) from biologic fluid sources, most commonly blood draws. Liquid biopsies from blood draws are appealing as they are relatively non-invasive and are already being used in select clinical settings, such as identification of treatment resistance in known EGFR mutation-positive non-small cell lung cancer patients. Research into the role of liquid biopsies for lung cancer diagnosis has demonstrated that tumor-derived cfDNA can potentially be detected in Stage IIB-IV disease. However, diagnosis of earlier stage disease remains limited given the low availability of tumor-derived DNA in the liquid biopsy samples.

While most of the liquid biopsy field has focused on increasing the analytical sensitivity of sequencing techniques, there remains the fundamental limitation that at a certain point the amount of tumor-derived DNA in the blood is so few or absent that detection cannot be overcome by sequencing. This raises the following question: how can the amount and fraction of tumor-derived DNA in liquid biopsy samples be increased so that the limitation in detecting early-stage cancer is overcome? While the exact mechanisms of cfDNA formation and clearance have not been fully elucidated, it is likely that the majority of cfDNA is produced by apoptotic cells, and these apoptotic cells and their associated cellular debris are rapidly cleared in the local microenvironment by tissue resident macrophages. As a consequence, out of the hundreds of millions of cells in a developing tumor, only picograms of tumor DNA can be found in the peripheral blood in stage I cancer. It is therefore proposed that sampling biologic fluid specimens in contact with the local tumor microenvironment and collecting the associated tissue-resident macrophages could allow one to acquire much more tumor-derived DNA than would be possible with liquid biopsies from blood samples.

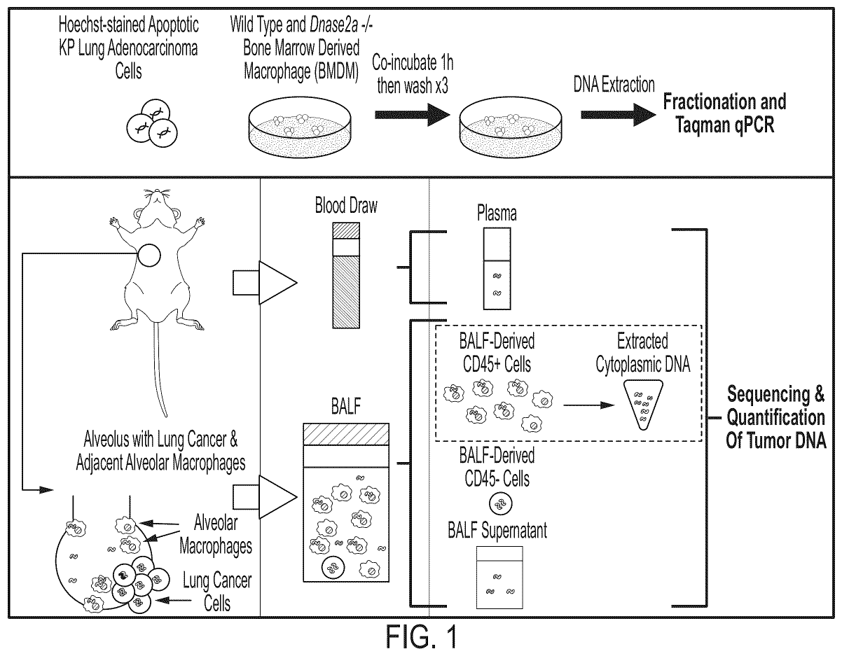

Lung cancer, particularly non-small cell lung cancer, provides a unique opportunity in that (1) there is a clear need for improved diagnostics, (2) there are methods of acquiring liquid biopsy samples more proximal to the tumor microenvironment than blood using either minimally invasive techniques (diagnostic bronchoscopy with bronchoalveolar lavage) or non-invasive techniques (induced sputum), and (3) these samples contain local tissue resident macrophages. BALF enables large volume sampling of the local microenvironment and collection of numerous tissue resident alveolar macrophages (human BALF typically contains 100,000-150,000 cells/mL with 80-90% of these cells being alveolar macrophages). While there are small cohorts that have demonstrated the ability to detect select driver mutations more frequently in BALF compared to blood, the baseline detectability of tumor-derived cfDNA using more advanced sequencing techniques has been largely unexplored. Furthermore, extraction of tumor-derived DNA from tissue resident macrophages for use in liquid biopsies has not been previously described and would be a novel approach that could unlock a potentially large, untapped resource in cancer diagnostics. As described herein, using in vitro and in vivo models, a method has been developed to extract tumor-derived DNA from macrophages for sequencing, and to determine the baseline of detection for tumor-derived DNA from alveolar macrophages (FIG. 1). This technology provides the foundation for additional translational experiments directed at further enhancing detectability of early-stage lung cancer.

### Example 2—Macrophages Engulf and Digest DNA from Tumor Cells

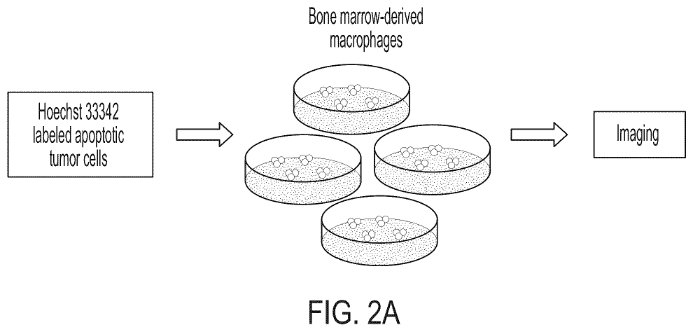

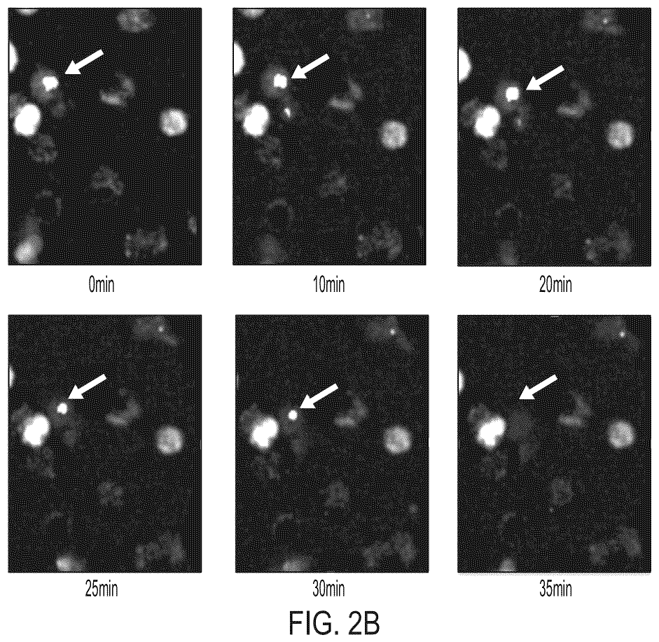

Because dying tumor cells are cleared in vivo by macrophages, it was hypothesized that engulfment of tumor DNA by macrophages could be recapitulated using an in vitro cell culture system. Hoechst-labeled apoptotic K-rasLSL-G12D/+; p53fl/fl (KP) lung adenocarcinoma (KPLG) cells were incubated with murine bone marrow-derived macrophages (BMDM) (FIG. 2A). Using fluorescent imaging, Hoechst-positive puncta were observed in the cytoplasm of BMDMs (FIG. 2B). The Hoechst-positive puncta were digested inside BMDMs (FIG. 2B), indicating that inhibiting DNA-digestion pathways could potentially increase the level of tumor DNA engulfed by macrophages, thereby enhancing the limit of detection in downstream sequencing applications.

### Example 3—Effect of DNase II Knockdown on Recovery of Tumor DNA in Macrophages In Vitro

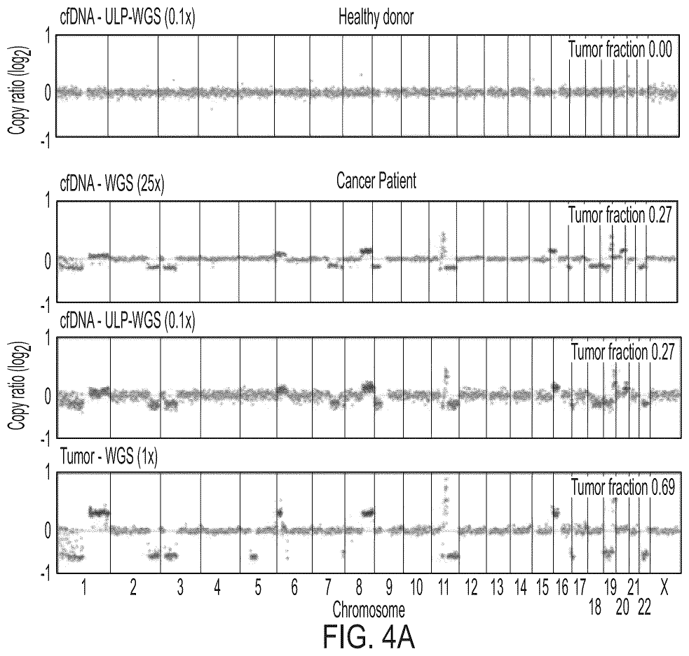

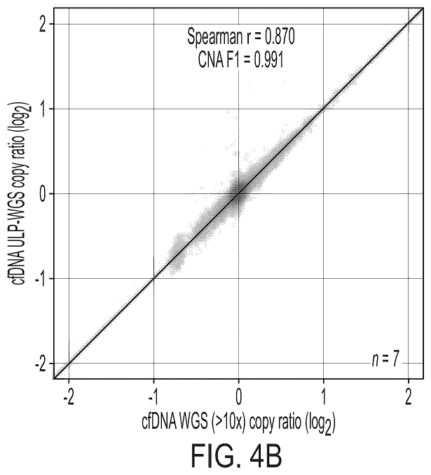

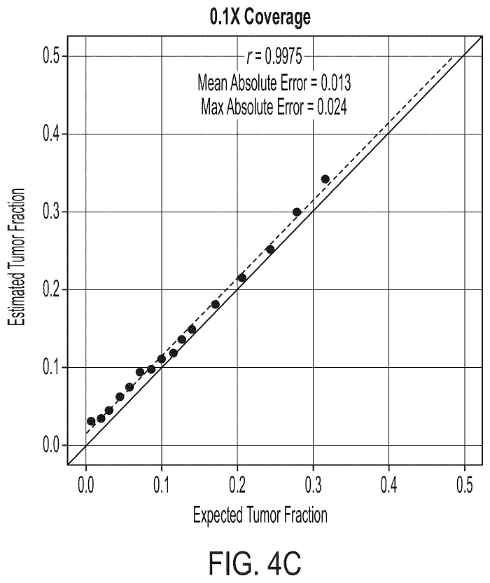

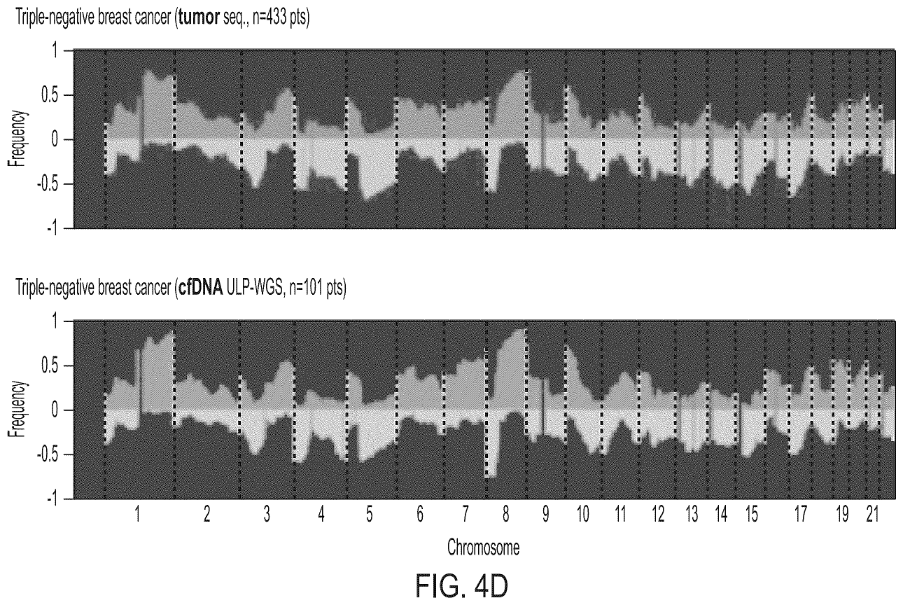

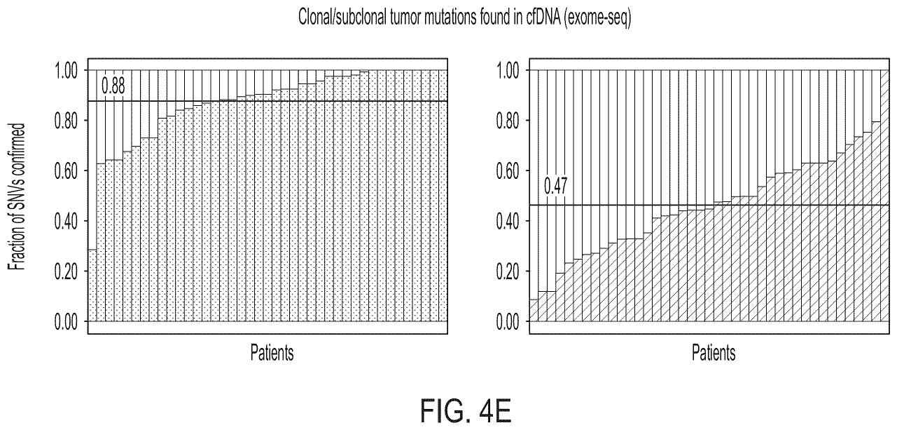

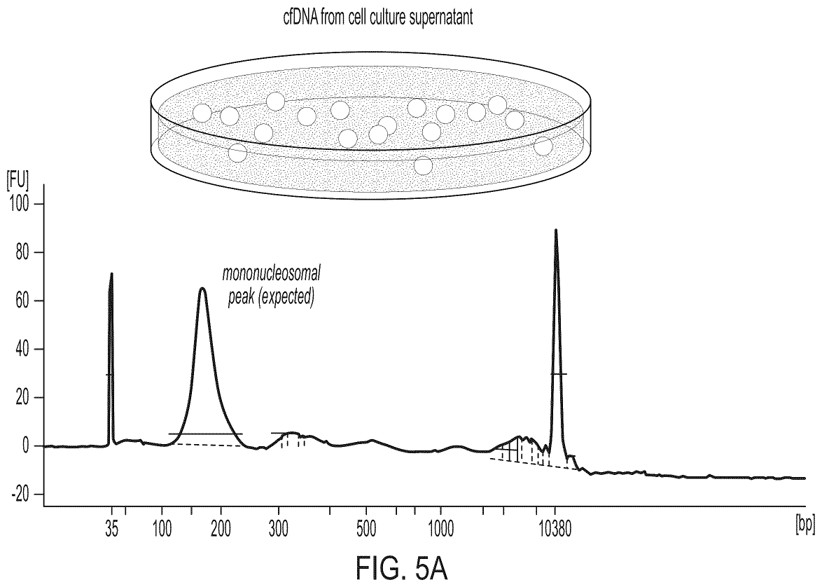

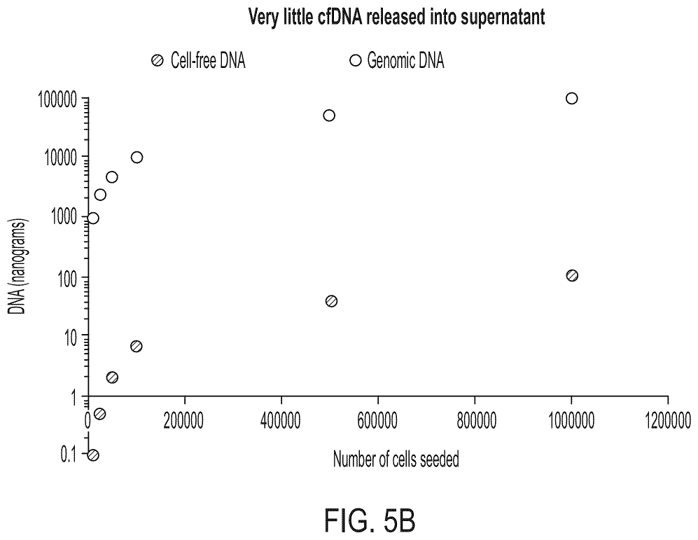

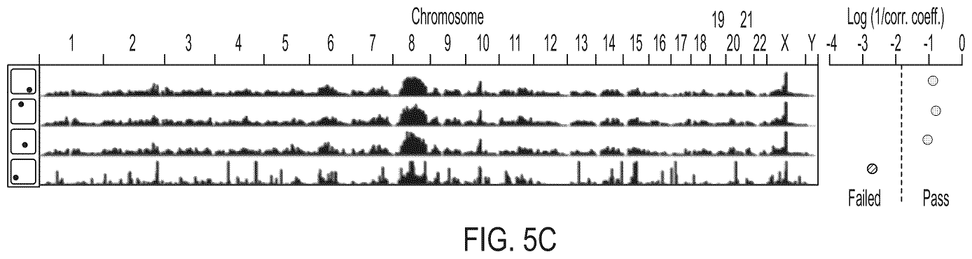

It is well established that DNase II, also referred to as DNase 2a, is a major DNA-degrading nuclease responsible for lysosomal digestion of DNA in macrophages. Multiple in vitro and in vivo studies have shown that DNase II knockout (−/−) leads to accumulation of ingested DNA in macrophage lysosomes^{34,35}. The accumulated DNA is likely to reflect cell-free DNA in size and fragmentation pattern according to nucleosome occupancy of the originating cell type. This suggests that existing methods for cell-free DNA analysis ought to be directly compatible^{29,30}, and could perhaps be augmented by administration of agents that inhibit DNase II activity (FIGS. 3 and 4A-4E). Therefore, it is expected that significantly more tumor DNA could be recovered from DNase II−/− macrophages, which model DNase II inhibition, than could be recovered by apoptotic cells directly. For instance, less than 0.1% of the genomic DNA from a cancer cell line can be recovered as cell-free DNA after 96 hours (FIGS. 5A-5B)—which is an extreme upper bound for what would be observed in vivo, considering cell-free DNA is cleared from the bloodstream within minutes³⁷. Tumor DNA can be expected to accumulate within macrophages (e.g., BMDMs) and be protected from extracellular nucleases. In addition, if each macrophage engulfs an entire cancer cell, it could be feasible to purify entire cancer genomes from macrophages using methods for single-cell isolation and sequencing^{38,39} (FIG. 5C). This could provide significant advantages over existing cell-free DNA tests.

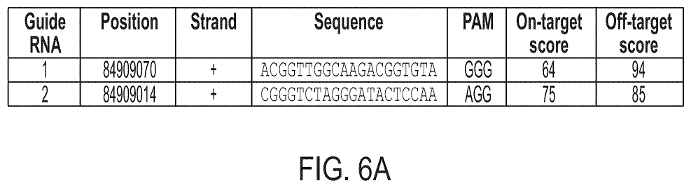

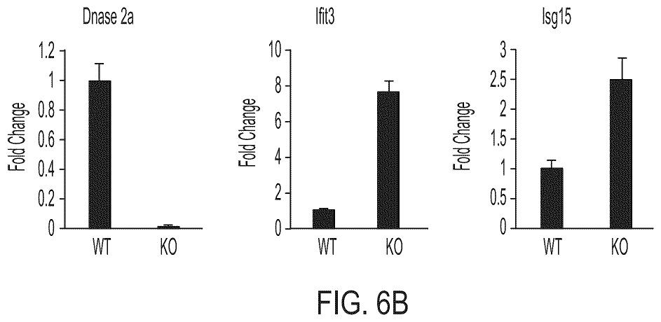

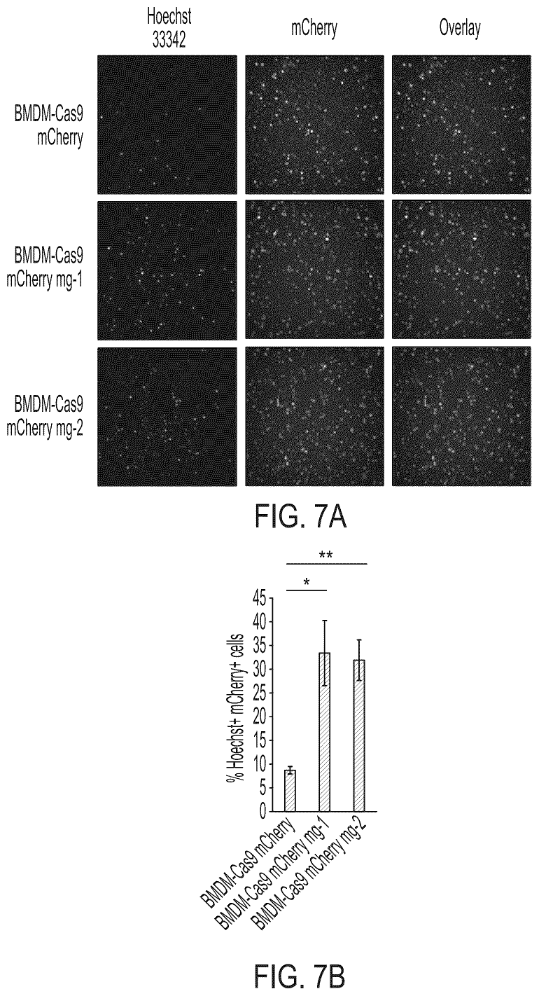

To test the effect of DNase II inhibition on recovery of tumor DNA, a matched set of wild type (WT) and DNase II−/− (KO) macrophages was generated using well-established methods for nucleofection and CRISPR knockout⁴⁰. Briefly, two different guide RNAs were used to knock out DNase II expression in BMDM using CRISPR (FIG. 6A). Knockout of DNase II in BMDM resulted in depletion of DNase II mRNA and the upregulation of genes such as Ifit3 and Isg5, as previously reported (FIG. 6B). Co-culture was then established with Hoechst-labeled KrasLSL-G12D/WT; p53FL/FL (KP) lung cancer cells⁴¹ (KPLG) and either wild-type control BMDMs or DNase II KO BMDMs. An increased accumulation of Hoechst-positive puncta in DNase I KO BMDMs was observed, as compared to the wild-type control cells (FIGS. 7A and 7B).

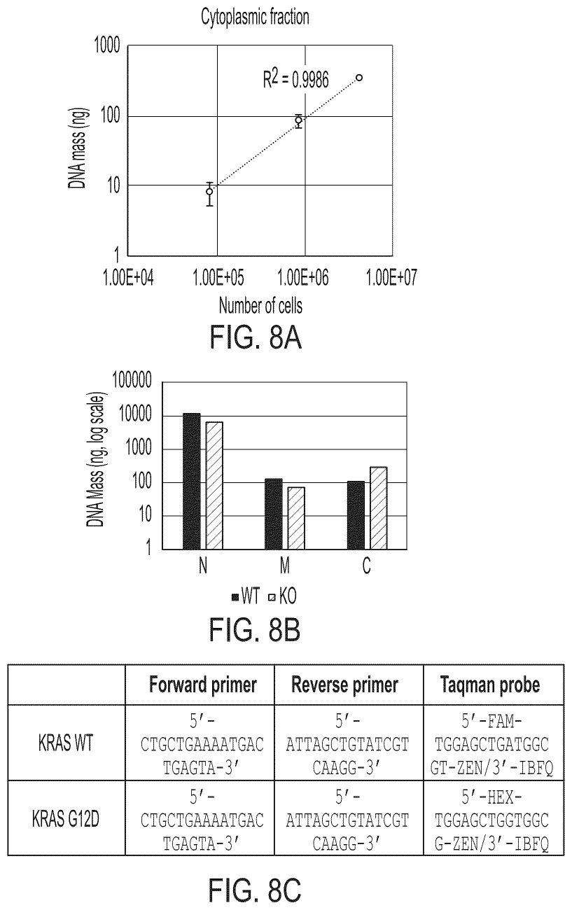

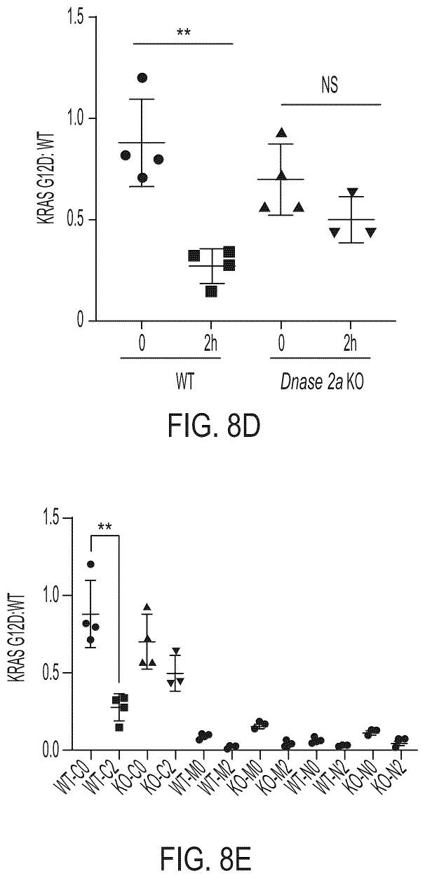

As most of the ingested DNA is thought to be localized within endosomes and lysosomes in the cytosolic compartment of macrophages, extracted macrophage DNA was fractionated into cytosolic, nuclear, and mitochondrial fractions using previously published methods. The yield, size distribution, and tumor fraction of DNA fractions is then compared versus total cell-free DNA in the culture supernatant. Cytoplasmic DNA could be recovered from macrophage cells co-incubated with apoptotic KPLG cells (FIG. 8A). Interestingly, in DNase II KO BMDMs more DNA from the cytoplasmic fraction as compared to wild-type control BMDMs, but not from the nuclear or the mitochondrial fractions (FIG. 8B). The total cytoplasmic DNA mass showed a linear correlation with input cell number and was within the range of macrophages quantities that could reasonably be recovered from each alveolar lavage procedure conducted in a mouse (FIG. 8A).

Next, it was tested whether washing away apoptotic KPLG cells would result in loss of tumor DNA signal inside the cytoplasmic part of BMDM cells, either in control cells or DNase II KO cells. As KRAS G12D is a hallmark mutation in KPLG cells, TaqMan quantitative PCT (qPCR) for KRAS G12D was used to specifically distinguish the tumor DNA from normal DNA, which encodes wild-type (WT) KRAS (FIG. 8C). In wild-type macrophages, the G12D signal dropped significantly within a 2-hour time scale. However, DNase II KO macrophages failed to clear KRAS G12D efficiently (FIG. 8D). For DNA recovered from other cellular compartments, the overall KRAS G12D DNA levels were low (FIG. 8E). Together, these data indicate that degradation of engulfed tumor DNA is mediated by DNase II and that inhibition of DNase II could increase tumor DNA signal recovered from the cytoplasmic part of macrophages.

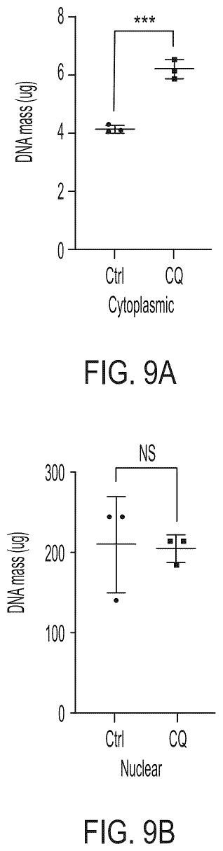

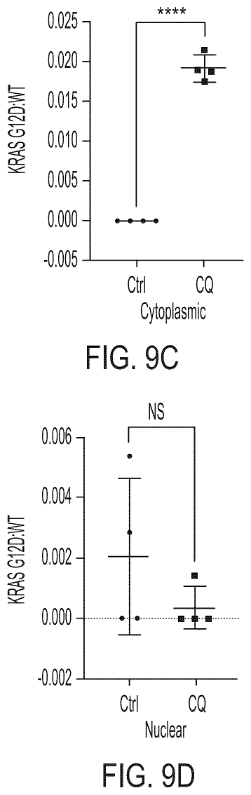

To test if pharmacologically blocking the DNA degradation pathway could lead to increased recovery of tumor DNA in macrophages, apoptotic KPLG cells were co-incubated with macrophage cells for 1 hour, after which apoptotic cells were washed away, and macrophages were treated either with or without 25 μM chloroquine. Chloroquine treatment was observed to increase total cytoplasmic DNA mass as well as the presence of KRAS G12D in the cytoplasmic DNA fraction of macrophages (FIGS. 9A-9D). These results further confirm that blocking the DNA degradation pathway, whether by inhibiting the activity of DNase II directly or indirectly, results in an increase in cytoplasmic DNA that can be recovered from macrophages.

In addition to the studies above, the impact on yield of apoptotic index of the cancer cell line, cancer cell to macrophage ratio, incubation time, and macrophage polarization state (e.g., M1 vs. M2) may further be evaluated. Cytokine arrays may be used to monitor the pro-inflammatory versus anti-inflammatory state of macrophages before versus after co-culture. Ways to maximize efferocytosis of cancer cells such as blocking SIPRa42, CD4742, or the MER tyrosine kinase⁴³, and/or treating with anti-tumor antibodies⁴⁴ may be further explored. Lysosomal DNA may be sequenced and analyzed to confirm that the same tumor genetic profile can be obtained as can be obtained directly from the cancer cell line, including copy number alterations (FIG. 2), nucleosome-imprinted fragmentation profiles⁴⁵, DNA methylation⁴⁶, and somatic mutations⁴⁷. Sequencing of lysosomal DNA from single macrophages can further be assessed by adapting previously established single-cell methods³⁸ to determine whether entire cancer genomes are recoverable from single macrophages (FIG. 3C).

Based on the results described above, it is expected that DNase II−/− macrophages will accumulate large amounts of tumor DNA in their lysosomes when co-cultured with tumor cells, and that the tumor DNA can be recovered and sequenced, and will reflect the genetics of the cancer cell line. It is further expected that macrophages could be reprogrammed by cancer cells, so their polarization state and phagocytic activity can also be monitored. It is also possible to enhance phagocytic function. However, it is expected that macrophages with higher phagocytic activity may be more adherent, which could present challenges when seeking to recover them in future in vivo experiments. Thus, attenuation of macrophage adherence while retaining phagocytic activity may also be desired.

### Example 4—Alveolar Macrophages Demonstrate Increased Sensitivity to Tumor DNA In Vivo

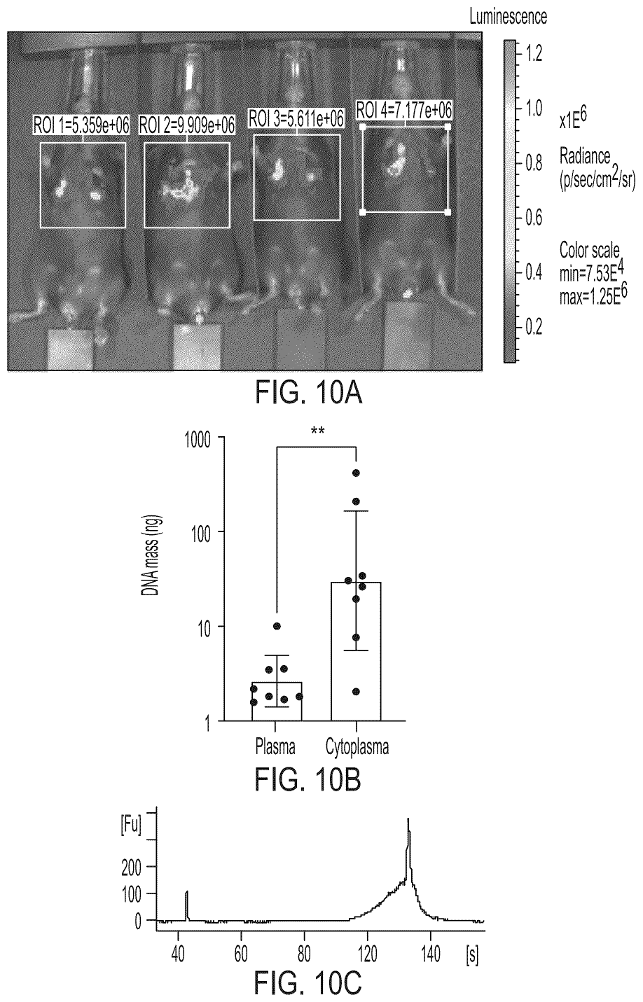

Having demonstrated that tumor DNA signal can be detected in the cytoplasmic fraction of macrophages co-incubated with tumor cells, it was hypothesized that in lung cancer mouse models, cancer mutations could also be detected in the cytoplasmic DNA of lung alveolar macrophages. KPLG cancer cells were inoculated into C57BL/6J mice through tail vein injection and tumor cell homing and growth was monitored through luciferase imaging of labeled cancer cells (FIG. 10A). Once tumors formed and luminescent signals were greater than 1E7, plasma and BALF samples were collected. CD45+ cells were isolated from the BALF, from which cytoplasmic DNA was further isolated. The total DNA mass recovered from the cytoplasmic fraction of the CD45+ cells was significantly more than the DNA mass recovered from the plasma (FIG. 10B). Notably, the cytoplasmic DNA from the CD45+ lung alveolar cells mainly consisted of large fragments, ranging from thousands to tens of thousands base pairs long (FIG. 10C).

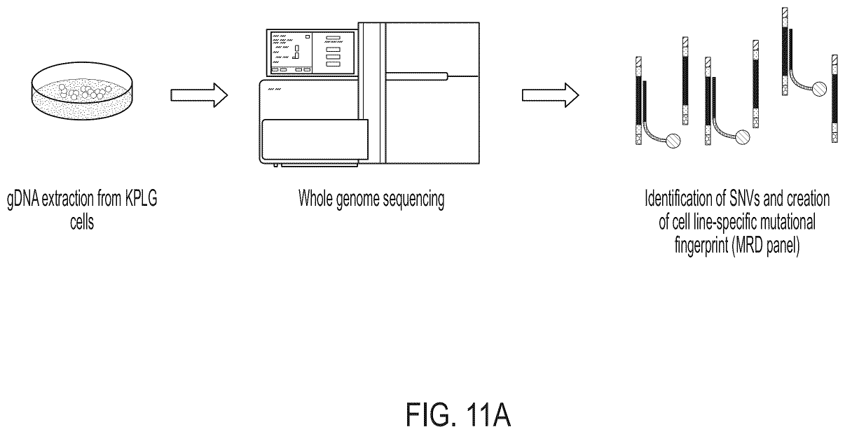

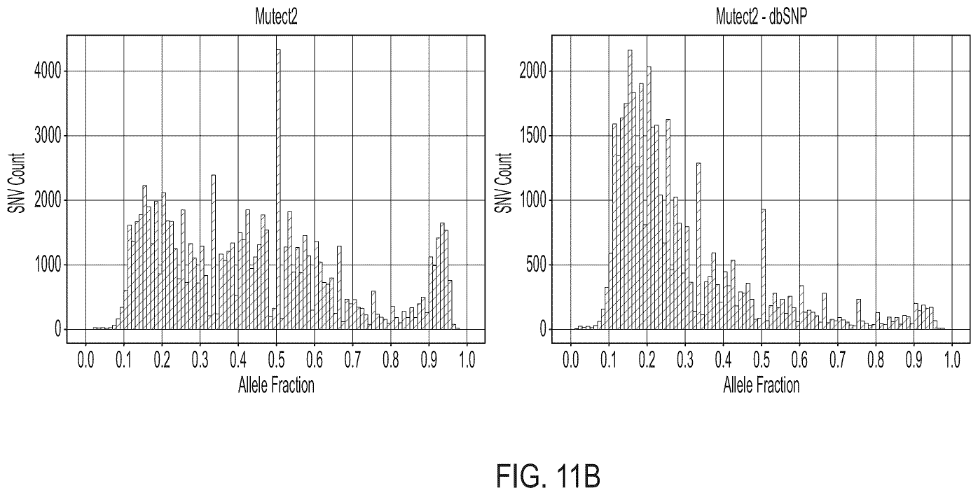

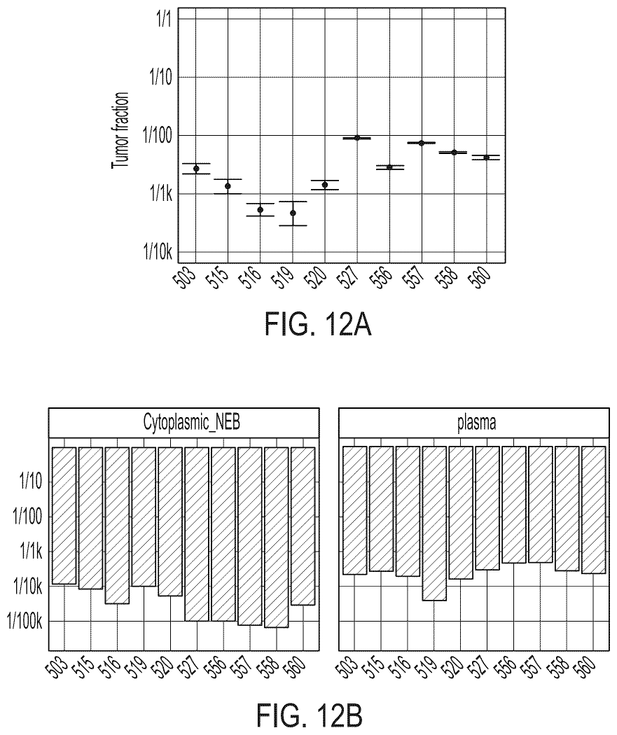

A mutation fingerprint detection assay was then designed to track 999 mutations, utilizing duplex-sequencing for error suppression⁶⁸. This assay could reliably detect single mutant duplexes and quantify tumor fractions down to 1E-4 (FIGS. 11A and 11B). Tumor DNA was detected in all cytoplasmic DNA samples from KPLG-lung adenocarcinoma-bearing mice (FIG. 12A), whether cells were sorted with anti-CD45 staining (first 5 mice) or were unsorted (last 5 mice). Cytoplasmic DNA recovered from BALF cells, either CD45+ or unsorted, showed an improvement in the detection limit as compared to the detection limit of cfDNA collected from the plasma (FIG. 12B). These results indicate that alveolar macrophages can be effectively utilized to collect DNA produced by apoptotic cells in developing lung tumors and that the phagocytosed DNA can be assessed more deeply than cfDNA collected through standard liquid biopsies.

Additionally, it should be noted that the tumor fraction of DNA collected from macrophages in the presence of naturally occurring lung cancers may be even lower. Perhaps due to inoculation of the mouse model through tail vein injection, many KPLG tumors were observed to develop in deeper lung tissue, rather than in the airway epithelium. This phenomenon would likely shift a portion of the DNA generated by apoptotic cancer cells away from alveolar macrophages and towards the bloodstream. For this reason, lung cancers that develop at a primary site in the airway, particularly non-small cell lung cancers that are in close association with alveolar macrophages, should have a substantially lower detection limit and higher tumor fraction in alveolar macrophage cytoplasmic DNA than the mouse model presented herein. Even so, it is encouraging that alveolar macrophages from this mouse model could be used to successfully detect lung tumors originating from an extant site. As discussed above, detection limit and tumor fraction in alveolar macrophage cytoplasmic DNA could also be enhanced by pretreatment with agents that inhibit the activity of nucleases that digest phagocytosed tumor DNA, namely DNase II.

### Example 5—Effect of DNase II Knockdown on Recovery of Tumor DNA in Macrophages In Vivo

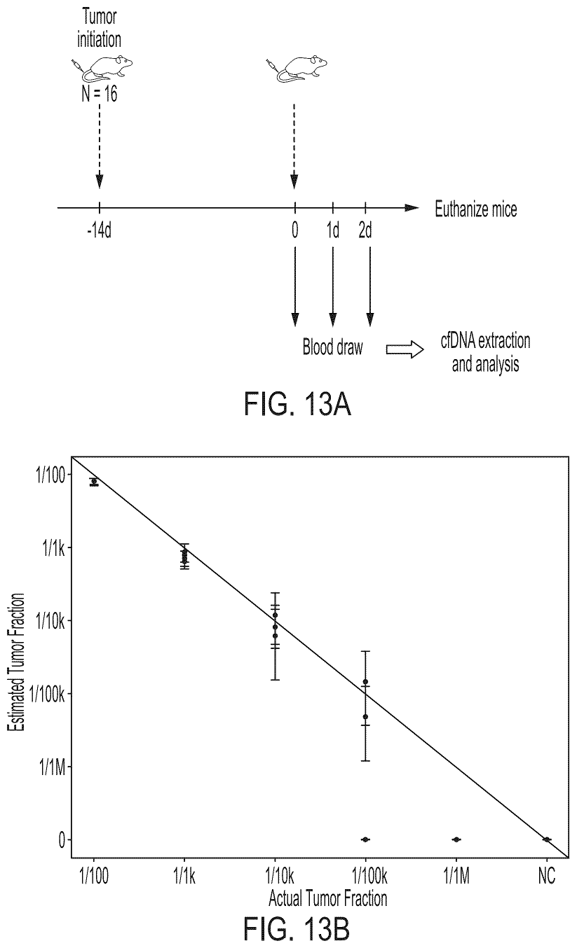

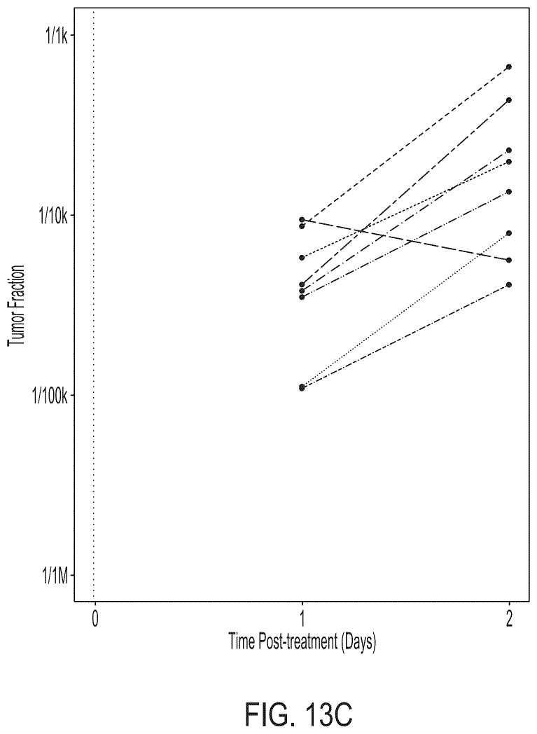

Whether or not the same effect can be achieved in vivo using the pharmacologic inhibition described in Example 3, tumor-bearing mice may be studies using DNase II KO macrophages instilled onto the airways. Adoptive transfer of genetically modified macrophages has been described in multiple studies⁵⁰, while instilled macrophages have been shown to traffic to tumors⁵¹. Lung adenocarcinoma, one of the main subtypes of lung cancer, often develops at the level of the terminal respiratory bronchioles and alveoli in peripheral lung fields. Clara and alveolar type II cells, which are found in terminal respiratory bronchioles and alveoli respectively, have been implicated as potential cells of origin for peripheral based lung adenocarcinoma. The current paradigm for lung adenocarcinoma tumorigenesis is thought to involve progression from pre-invasive lesions, such as atypical adenomatous hyperplasia and adenocarcinoma in situ, to subsequent invasive tumors. Early pre-invasive lesions generally develop along the alveolar and airway walls in what is known as a lepidic pattern of growth, consistent with this paradigm for adenocarcinoma tumorigenesis. As a result, alveolar macrophages are often in close proximity to the site of lung adenocarcinoma formation. Hundreds of thousands to millions of alveolar macrophages are recovered by sputum induction a simple outpatient procedure where nebulized saline is used to induce sputum production- or by bronchoalveolar lavage³⁶ (BAL). The aforementioned strategies to enhance efferocytosis could further improve diagnostic yield. Cell-free DNA from tumor-bearing mice has previously been isolated and sequenced and can be compared to tumor DNA recovery from plasma, BAL fluid, and DNase II KO macrophages, using ultrasensitive methods previously established²⁹ (FIGS. 13A-13C).

Well-established protocols are followed for (i) adoptive transfer of syngeneic WT and KO macrophages via intratracheal administration⁵² in C57BL/6 mice bearing orthotopic KP lung cancer xenograft, and (ii) bronchoalveolar lavage (BAL) in mice⁵³. A near infrared dye is used that allows in vivo cell tracking to monitor the distribution of instilled macrophages before and after BAL. The fraction and viability of recoverable macrophages is assessed, as well as techniques to maximize the recovery of instilled macrophages via BAL. An orthotopic lung cancer model is then developed using luciferase-expressing KP mouse lung cancer cells in C57BL/6 mice^{54,55}. Following tumor establishment, the adoptive transfer and BAL steps are repeated. In vivo luciferase and fluorescent imaging are used to monitor tumor formation and macrophage localization. The yield and tumor fraction of lysosomal DNA recovered from WT and KO macrophages are compared with cell-free DNA recovered from BAL fluid and peripheral blood. Cytology is performed on BALF. Ultra-low pass whole genome sequencing (ULP-WGS) and targeted sequencing for hundreds of mutations present in KP lung cancer cells are then conducted. The former approach enables cancer detection without prior knowledge of the mutations in the tumor (FIGS. 4A-4E), while the latter approach can detect extremely low levels of tumor DNA when the tumor is known, e.g., 1/100,000 tumor fraction from just 20 ng of fragmented DNA (FIGS. 13A-13C). The amount of tumor DNA recovered is subsequently correlated with tumor volume. Tissue histology is utilized to examine macrophage infiltration into the tumor bed. Other concurrent treatments are concurrently explored to promote efferocytosis of tumor cells by macrophages.

It is expected that one would find that at least some fraction of instilled macrophages will be retrievable, hours to days after adoptive transfer, and that much more tumor DNA will be recoverable from DNase II KO macrophages than from WT macrophages or the cell-free DNA in peripheral blood or BAL fluid. However, little is known about the biomechanics of macrophages and tumor cells in the alveolus. The surface area of the alveolus is very large, and it could take time for instilled macrophages to locate the tumor. It is also possible that instilled macrophages could adhere to lung epithelium, or penetrate into the tumor bed, and not be recoverable via BAL³⁶. If this happens, ways to disrupt cell adhesion could be utilized, e.g., using lidocaine to enhance recovery. Ways to dislodge cancer cells during the BAL procedure could further enhance detection using BALF cytology. Another alternative strategy could be to leverage the homing capacity of macrophages for imaging-based detection. Macrophages could be engineered to secrete analytes or to alter their behavior in the tumor microenvironment⁵¹. DNase II KO macrophages would also be expected to activate interferon signaling which could promote anti-tumor activity and shrink tumors and/or cause unwanted inflammatory responses in the airways. If the former occurs, then the approach could become a theranostic; while if the latter occurs, one could pursue concurrent IFNAR knockout as previously described to abrogate type I IFN response³⁵. DNase II KO macrophages may accumulate DNA from other cell types, which may dilute the signal. It will be important to distinguish preinvasive from invasive lesions, as well as specific subtypes of lung cancer, and the feasibility of detecting each cancer subtype can be determined in distinct models based on unique genetic and epigenetic signatures⁵⁶.

### Example 6—Recovery of Tumor DNA from Macrophages Treated with DNase II Inhibitors or Interfering RNAs

Multiple DNase II inhibitors exist including peptides⁵⁷ and small molecules⁵⁸, while macrophages are tractable targets for gene therapy⁵⁹ given their propensity to ingest foreign particles. Intratracheal knockdown of genes using nano-encapsulated siRNAs has also been examined.

Microsphere-based alveolar macrophage-targeting particles containing the above-mentioned DNase II inhibitors and/or siRNAs may be generated. Microspheres containing the diluting vehicle may be used as a control. These microspheres may be initially tested in the in vitro co-culture system of Examples 2 and 3. The IC₅₀ and EC₅₀ of DNase II inhibitor-containing microspheres may be determined, as well as the optimal treatment duration. Inhibition of DNase II may be confirmed using DNA digestion assays. For microspheres containing siRNA, knockdown efficiency may be confirmed by western blotting. Whether treatment with DNase II inhibitors or siRNA-containing microspheres confers the same tumor DNA buildup effect in macrophages as DNase II knockout in the in vitro co-culture system may be evaluated to rule out potential off-target effects of these small molecules/siRNAs or of the microspheres themselves. The DNA uptake by the macrophages may be measured and quantified using the previously described methods.

Once the potency of DNase II inhibitor- or siRNA-containing microspheres is confirmed, DNase II inhibitor- or siRNA-containing microspheres may be tested in the lung-cancer mouse models of Examples 4 and 5. Aerosolized microspheres may be generated as these are classically known to be optimal for lung-targeted delivery and have been shown to be phagocytosed by alveolar macrophages. Alveolar macrophages are then isolated before and after treatment. Macrophage lysosomes and tumor DNA may be further isolated and used to compare the yield and tumor fraction of lysosomal DNA to that of BAL fluid and peripheral blood. The amount of recovered tumor DNA is then subsequently correlated with tumor volume, while tissue histology is utilized to examine macrophage infiltration.

It is expected that inhibition of DNase II will result in a much higher yield of tumor DNA from the macrophage lysosomes in lung cancer-bearing mouse models. It is possible that existing DNase II inhibitors and genetic knockdown strategies may either not provide the required specificity, or carry other limitations that affect their in vivo use. If this happens, high-throughput screening for other inhibitors of DNase II could be identified. Of note, aerosolized DNase II inhibitor/siRNA-containing microspheres could also be taken up by the cancer cells. In addition, inhibition of DNase II will have potential impact on the macrophage polarization status and the tumor microenvironment, which can alter the growth rate of the tumors. As such, tumor growth along the DNase II treatment time course using luciferase imaging, and record survival time of the mice can be conducted as well. In addition, the change of immune cells in the tumor microenvironment with or without DNase II inhibitor treatment by CyTOF can be monitored. Potential combination therapies will be further evaluated based on the results.

There exists a significant unmet need for better lung cancer diagnostics. Diagnosis of lung cancer at early-stage disease when surgery is potentially curative could substantially improve clinical outcomes and reduce lung cancer associated mortality. While lung cancer screening and the increased use of CT scans have led to the identification of millions of pulmonary nodules, one of the ongoing challenges is determining which of these nodules are cancerous and which are benign. Since the majority of pulmonary nodules are benign, providers often favor “watchful waiting” for most small nodules given the risk/benefits of biopsy or surgical resection. This approach unfortunately is predicated on allowing cancerous lesions to grow before intervening. The approach described herein aims to address the challenge of how to better and more expeditiously approach the diagnosis of these small pulmonary nodules by harnessing macrophages as collection vehicles to shuttle massive amounts of tumor DNA from deep within the lungs for analysis. This approach, which combines techniques for collecting BALF and induced sputum with sequencing-based diagnostics for pulmonary nodules, could improve lung cancer diagnostics in three major ways. First, it could improve diagnostic yield from bronchoalveolar lavage of peripheral nodules, where 70% of lung cancers develop. Second, by coupling it with sputum induction, it could help to clarify which of millions of indeterminate pulmonary nodules require invasive biopsy. Third, this approach could help to broaden lung cancer screening, by providing both a means to clarify the need for invasive follow-up after chest CT scanning, and possibly a new mechanism for initial screening, using at-home sputum collection. This approach could result in a more immediate clinical impact by substantiating methods to improve the diagnostic yield of an existing procedure, as well as providing the foundation for more long-term technological advancements that could significantly impact how we approach liquid biopsies and cancer diagnostics. In all, this approach could improve lung cancer screening, testing, and care for millions of patients.

This approach, in part, seeks to bridge the gap between the powerful sequencing technology used for liquid biopsy testing and the physical inaccessibility of tumor DNA in early-stage cancer. This approach focuses on lung cancer, but the concept of programming macrophages to ingest and shuttle material out of the body is novel in a broad sense and could be adapted for other challenging diagnostic applications as well. For instance, the approach could be used to diagnose other cancers, deep-seated infections, etc., from other fluids where macrophages are recoverable (e.g., from urine in bladder cancer, from stool in colon cancer, from blood in hematologic malignancies, etc.). This approach to cancer detection is through a different lens than most in that much of the liquid biopsy field has focused on improving analytical sensitivity, whereas the instant approach focuses more on the fundamental challenge that most tumor DNA is not shed into the peripheral blood. This strategy-harnessing macrophages as cellular collection vehicles-could establish an entirely new paradigm for the diagnostics field.

### Methods for Examples

Cell lines: KPLG cells were kindly provided by the Jacks Laboratory at the Massachusetts Institute of Technology (MIT). BMDM were harvested from femurs of wild-type C54BL/6 mice and DNase 2a−/− mice. KPLG cells were cultured in DMEM supplemented with 10% fetal bovine serum (Gibco) and 1% penicillin/streptomycin (Corning); BMDM cells were cultured in DMEM supplemented with 10% fetal bovine serum (Gibco) and 1% penicillin/streptomycin (Corning), and 15% L929 conditioned medium. All cells were cultured in a humidified atmosphere of 95% air and 5% CO₂ at 37° C. The KPLG cell line was declared pathogen free after being subjected to murine pathogen testing by the Diagnostic Laboratory of the Division of Comparative Medicine (MIT).

Imaging: KPLG cells were collected using 0.25% trypsin EDTA and apoptosis was induced by 1 μM staurosporin for 24 hours. The apoptotic cells were collected by centrifugation at 3000×g for 7 min. Cells were resuspended in 2 mL PBS, with 10 pg/mL Hoechst 33342, 10 min. at room temperature. The cells were collected by centrifugation at 3000×g for 7 min. The cells were washed with 15 mL PBS three times, and co-incubated with the macrophages for the indicated time. Images were taken using the Perkin Elmer Opera Phenix Imaging system.

Cell fractionation and DNA extraction: Cells were collected by centrifugation at 3000×g for 7 min. The cells were lysed using four volumes of lysis buffer (20 mM Tris-HCl, pH 7.4, 10 mM KCl, 2 mM MgCl2, 1 mM DTT, and 1 mM EDTA) for 20 min. on ice. The nuclear pellet was collected by centrifugation twice at 800×g for 5 min. The mitochondrial fraction was collected by centrifugation twice at 10,000×g for 10 min. The collected supernatant was used as the cytoplasmic fraction. The nuclear, mitochondrial, and cytoplasmic fractions in lysis buffer were incubated with Protease K at 56° C. for 10 min. DNA was extracted with DNA binding columns (QIAGEN DNA Mini Kit).

Tumor models and study design: All animal work was approved by the committee on animal care (MIT protocol 0420-023-23). 4-6 weeks old female C57BL/6J mice (Taconic Biosciences) were injected with 5×10⁵ KPLG cells resuspended in PBS (Gibco) via the tail vein. Tumors were monitored through luciferase imaging. Blood was collected under anesthesia retro-orbitally. 70 μL of blood were collected from alternating eyes with non-heparinized hematocrit capillary tubes and resuspended 1:1 (v/v) in 10 mM ethylenediaminetetraacetic acid (EDTA) in PBS. All blood samples were kept on ice and plasma separated within 2 hours of blood collection. Plasma was separated by centrifuging blood samples at 8,000×g for 5 min. at 4° C. and stored at −80° C. Bronchoalveolar lavage was performed following published protocols. Mouse lungs were also harvested and subjected to hematoxylin and eosin (H&E) staining.

Probe panel: A KPLG-specific probe panel was designed by selecting 999 heterozygous single nucleotide variants (SNVs) from a published list (Castle et al., BMC Genomics 2014).

cfDNA extraction and quantification: Frozen plasma was thawed and centrifuged at 15,000×g for 10 min. to remove residual cells and debris. 1×PBS was then added into plasma to make the total volume 2.1 mL for cfDNA extraction using the QIAsymphony Circulating DNA kit. The extracted cfDNA was quantified using a qPCR assay and then frozen at −20° C. until ready for further processing.

gDNA extraction and shearing: gDNA was extracted from KPLG cells using the QiaAmp DNA Mini Kit. The extracted gDNA was sheared to 150 bp in size using a Covaris LE 220 instrument. Sheared DNA was quantified using Qubit (Qubit™ dsDNA HS, Invitrogen).

Library construction, hybrid capture, and sequencing: cfDNA and gDNA libraries were constructed using the Kapa Hyper Prep Kit with custom dual index duplex UMI adapters (IDT). A maximum of 50 μL of extracted cfDNA or 20 ng cfDNA mass was used as input into library construction. The prepared libraries were then quantified using the Quant-iT PicoGreen assay on a Hamilton STAR-line liquid handling system. Hybrid capture (HC) using a KPLG specific panel was performed using the xGen hybridization and wash kit with xGen Universal blockers (IDT) using a protocol adapted from Schmitt et al. For MRD tests, libraries were pooled up to maximum 12-plex, with a library mass equivalent to 25 times DNA mass into LC for each sample, and 0.56 pmol/uL of a panel consisting of 120 bp long probes (IDT) targeting 999 KPLG specific mutations was applied. After the first round of HC, libraries were amplified by 16 cycles of PCR and then carried through a second HC but with half volumes of human Cot-1 DNA, xGen Universal blockers, and probes. Mouse Cot-1 DNA was preliminarily tested and no impact on assay performance was observed. After the second round of HC, libraries were amplified through 8-16 cycles of PCR, quantified, and pooled for sequencing (151 bp paired-end runs) with a targeted raw depth of 250,000× per site for 20 ng DNA input. Sequencing data was processed by a duplex consensus calling pipeline previously described (Parsons et al, 2020 CCR).

### REFERENCES FOR EXAMPLES

1. The National Lung Screening Trial Research Team. N. Engl. J. Med. 365, 395-409 (2011)
2. Goldstraw, P. et al., J. Thorac. Oncol. 11, 39-51 (2016)
3. Hirsch, F. R. et al., Lancet 389, 299-311 (2017)
4. de Koning, H. J. et al., N. Engl. J. Med. 382, 503-513 (2020)
5. Moyer, V. A. et al., Ann. Intern. Med. 160, 330-338 (2014)
6. Asshoff, M. et al., Magazine of European Medical Oncology vol. 12 162-165 (2019)
7. Wallace, M. J. et al. Radiology 225, 823-828 (2002)
8. Baaklini, W. A. et al. Chest 117, 1049-1054 (2000)
9. Paone, G. et al. Chest 128, 3551-3557 (2005)
10. Muioz-Largacha, J. A. et al., J. Thorac. Dis. 9, S98-S103 (2017)
11. Lang, D. et al., Wien. Klin. Wochenschr. 130, 288-292 (2018)
12. Wang Memoli, J. S. et al., Chest 142, 385-393 (2012)
13. Bezel, P. et al., Clin. Lung Cancer 17, e151-e156 (2016)
14. Ost, D. E. et al., Am. J. Respir. Crit. Care Med. 193, 68-77 (2016)
15. Nair, V. S. et al. Am Thoracic Society (2018)
16. Guida, F. et al., JAMA Oncol 4, e182078-e182078 (2018)
17. Meyer, M. G. et al., Cancer Cytopathol. 123, 512-523 (2015)
18. Phillips, M. et al., Chest 123, 2115-2123 (2003)
19. Kirkpatrick, J. D. et al. Sci. Transl. Med. 12, (2020)
20. AEGIS Study Team. J. Natl. Cancer Inst. 109, (2017)
21. Hassanein, M. et al., Cancer Prev. Res. 5, 992-1006 (2012)
22. Calabrese, F. et al., J. Clin. Med. Res. 8, (2019)
23. Mazzone, P. J. et al., Am. J. Respir. Crit. Care Med. 196, e15-e29 (2017)
24. Seijo, L. M. et al., J. Thorac. Oncol. 14, 343-357 (2019)
25. Billatos, E. et al., Clin. Cancer Res. 24, 2984-2992 (2018)
26. Couraud, S. et al., European Journal of Cancer vol. 48 1299-1311 (2012)
27. Newman, A. M. et al., Nature Medicine vol. 20 548-554 (2014)
28. Bettegowda, C. et al., Sci. Transl. Med. 6, 224ra24 (2014)
29. Parsons, H. A. et al., Clin. Cancer Res. 26, 2556-2564 (2020)
30. Adalsteinsson, V. A. et al., Nat. Commun. 8, 1324 (2017)
31. Törmänen, U. et al., Cancer Res. 55, 5595-5602 (1995)
32. The Cancer Genome Atlas Research Network. Nature vol. 511 543-550 (2014)
33. Werfel, T. A. et al., Semin. Immunopathol. 40, 545-554 (2018)
34. Krieser, R. J. et al., Cell Death Differ. 9, 956-962 (2002)
35. Rodero, M. P. et al., Nat. Commun. 8, 2176 (2017)
36. Bhattacharya, J. et al., Semin. Immunopathol. 38, 461-469 (2016)
37. Kustanovich, A. et al., Cancer Biol. Ther. 20, 1057-1067 (2019)
38. Lohr, J. G. et al., Nat. Biotechnol. 32, 479-484 (2014)
39. Zhang, C.-Z. et al., Nat. Commun. 6, 6822 (2015)
40. Freund, E. C. et al., Journal of Experimental Medicine vol. 217 (2020)
41. DuPage, M. et al., Nature Protocols vol. 4 1064-1072 (2009)
42. Kojima, Y. et al., Nature 536, 86-90 (2016)
43. Vaught, D. B. et al., Cancer Cell Microenviron 2, (2015)
44. Trivedi, S. et al., Annals of Oncology vol. 26 40-47 (2015)
45. Cristiano, S. et al., Nature 570, 385-389 (2019)
46. Shen, N. et al., Front. Oncol. 9, 1281 (2019)
47. Chabon, J. J. et al., Nature 580, 245-251 (2020)
48. McWhorter, F. Y. et al., Cell. Mol. Life Sci. 72, 1303-1316 (2015)
49. Stover, D. G. et al., J. Clin. Oncol. 36, 543-553 (2018)
50. Suzuki, T. et al., The Keio Journal of Medicine vol. 64 51-51 (2015)
51. Aalipour, A. et al., Nat. Biotechnol. 37, 531-539 (2019)
52. Liu, H. et al., Cell Death Dis. 10, 664 (2019)
53. Sun, F. et al., BIO-PROTOCOL vol. 7 (2017)
54. LaFave, L. M. et al., Cancer Cell 38, 212-228.e13 (2020)
55. Manchado, E. et al., Nature 534, 647-651 (2016)
56. Chen, H. et al., Nat. Commun. 10, 5472 (2019)
57. Sperinde, J. J. et al., J. Gene Med. 3, 101-108 (2001)
58. Ross, G. F. et al., Gene Ther. 5, 1244-1250 (1998)
59. Ng, B. et al., Mol. Ther. Nucleic Acids 16, 194-205 (2019)
60. Kwon, E. J. et al., ACS Nano 10, 7926-7933 (2016)
61. Lo, J. H. et al., Mol. Cancer Ther. 17, 2377-2388 (2018)
62. Saung, M. T. et al., Small 12, 5873-5881 (2016)
63. Adalsteinsson, V. A. et al., Integr. Biol. 5, 1272-1281 (2013)
64. Ladas, I. et al., Clin. Chem. 63, 1605-1613 (2017)
65. Choudhury, A. D. et al., JCI Insight 3, (2018)
66. Parikh, A. R. et al., Nat. Med. 25, 1415-1421 (2019)
67. Manier, S. et al., Nat. Commun. 9, 1691 (2018)
68. Xiong, K. et al, Nucleic Acids Res. 50(1), el (2022)

### INCORPORATION BY REFERENCE

All patents and other publications, including documents, references, published patents, published patent applications, and co-pending patent applications cited throughout this application are, for example, described in the technology described herein. For purposes of describing and disclosing the methodology described in such publications that may be used in connection with the above, expressly incorporated herein by reference. These publications are provided solely for their disclosure prior to the filing date of the present application. Nothing in this regard should be construed as an admission that the inventors have no right to advance the date of such disclosure, based on the prior invention or for any other reason. All statements regarding the date or content of these documents are based on information available to the Applicant and do not give any approval as to the accuracy of the date or content of these documents.

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

What is claimed is:

1. A method for increasing the concentration of tumor DNA in a macrophage or population thereof, comprising administering to a subject an effective amount of an agent that results in an increase in the accumulation of tumor DNA in the macrophage or population thereof in the subject.

2. The method of claim 1, wherein the concentration of tumor DNA in the macrophage or population thereof is increased by up to 2-fold, up to 3-fold, up to 4-fold, up to 5-fold, up to 10-fold, up to 25-fold, up to 50-fold, up to 100-fold, up to 200-fold, up to 300-fold, up to 400-fold, up to 500-fold, up to 600-fold, up to 700-fold, up to 800-fold, up to 900-fold, or up to 1000-fold.

3. The method of claim 1 or 2, wherein the agent results in a reduction in the rate of tumor DNA degradation in the macrophage or population thereof.

4. The method of claim 3, wherein the rate of tumor DNA degradation in the macrophage or population thereof is reduced by up to 5%, up to 10%, up to 15%, up to 20%, up to 25%, up to 30%, up to 35%, up to 40%, up to 45%, up to 50%, up to 55%, up to 60%, up to 65%, up to 70%, up to 75%, up to 80%, up to 85%, up to 90%, up to 95%, up to 99%, or up to 100%, as compared to the rate of tumor DNA degradation in the macrophage or population thereof in the absence of the agent.

5. The method of any one of claims 1-4, wherein the agent is a small molecule that reduces the expression and/or activity of one or more deoxyribonucleases in the macrophage or population thereof.

6. The method of claim 5, wherein the agent is a DNase II inhibitor.

7. The method of claim 5, wherein the agent is a small molecule that results in an increase in the lysosomal pH of the macrophage or population thereof.

8. The method of claim 7, wherein the agent is chloroquine or a derivative thereof.

9. The method of any one of claims 1-4, wherein agent is a nucleic acid that reduces the expression and/or activity of one or more deoxyribonucleases in the macrophage or population thereof.

10. The method of claim 9, wherein the agent is an siRNA or shRNA that selectively binds to a mRNA transcript encoding DNase II.

11. The method of any one of claims 1-10, wherein the agent is administered intravenously, orally, or through inhalation.

12. The method of any one of claims 1-11, wherein the increased concentration of tumor DNA in the macrophage or population thereof is due to an increased concentration of tumor DNA in the lysosomes of the macrophage or population thereof.

13. The method of any one of claims 1-12, wherein the subject is a human patient.

14. The method of claim 13, wherein the subject has, is suspected of having, or is at risk for a disease.

15. The method of claim 14, wherein the disease is a cancer.

16. The method of claim 15, wherein the cancer is selected from: colorectal cancer, lung cancer, breast cancer, pancreatic cancer, prostate cancer, bladder cancer, kidney cancer, thyroid cancer, uterine cancer, cervical cancer, ovarian cancer, testicular cancer, esophageal cancer, stomach cancer, liver cancer, brain cancer, peritoneal cancer, lymphoma, leukemia, multiple myeloma, neuroblastoma, osteosarcoma, and soft tissue sarcoma.

17. The method of any one of claims 1-16, further comprising collecting the macrophage or population thereof.

18. The method of claim 17, further comprising isolating the tumor DNA from the macrophage or population thereof.

19. The method of claim 17, further comprising isolating the lysosomes from the macrophage or population thereof.

20. The method of claim 19, further comprising isolating the tumor DNA from the lysosomes.

21. The method of any one of claims 17-20, further comprising attenuating macrophage adherence, thereby increasing the yield of said macrophage or population thereof.

22. The method of any one of claims 17-21, further comprising increasing efferocytosis of tumor DNA by the macrophage or population thereof.

23. The method of any one of claims 17-22, comprising detecting the tumor DNA.

24. The method of any one of claims 1-23, wherein the agent is administered by inhalation as a formulation comprising aerosolized microspheres comprising said agent.

25. A method for increasing the recovery of tumor DNA from a macrophage or population thereof, comprising:
(a) administering to a subject an effective amount of an agent that increases the accumulation of tumor DNA in a macrophage or population thereof in a subject; and
(b) obtaining the tumor DNA from the macrophage or population thereof.

26. The method of claim 25, wherein the tumor DNA accumulates within the lysosomes of the macrophage or population thereof.

27. The method of claim 25 or 26, wherein the tumor DNA is obtained from the lysosomes of the macrophage or population thereof.

28. The method of any one of claims 25-27, wherein the macrophage or population thereof is collected in one or more biological samples.

29. The method of claim 28, wherein the one or more biological samples is a biological fluid.

30. The method of claim 29, wherein the one or more biological fluids comprise blood, urine, saliva, mucus, or sputum.

31. The method of any one of claims 25-30, wherein the concentration of the tumor DNA is increased by up to 2-fold, up to 3-fold, up to 4-fold, up to 5-fold, up to 10-fold, up to 25-fold, up to 50-fold, up to 100-fold, up to 200-fold, up to 300-fold, up to 400-fold, up to 500-fold, up to 600-fold, up to 700-fold, up to 800-fold, up to 900-fold, or up to 1000-fold.

32. The method of any one of claims 25-31, wherein the agent results in a reduction in the rate of tumor DNA degradation in the macrophage or population thereof.

33. The method of claim 32, wherein the rate of tumor DNA degradation in the macrophage or population thereof is reduced by up to 5%, up to 10%, up to 15%, up to 20%, up to 25%, up to 30%, up to 35%, up to 40%, up to 45%, up to 50%, up to 55%, up to 60%, up to 65%, up to 70%, up to 75%, up to 80%, up to 85%, up to 90%, up to 95%, up to 99%, or up to 100%, as compared to the rate of tumor DNA degradation in the absence of the agent.

34. The method of any one of claims 25-33, wherein agent is a small molecule that reduces the activity of one or more deoxyribonucleases in the macrophage or population thereof.

35. The method of claim 34, wherein the agent is a DNase II inhibitor.

36. The method of claim 34, wherein the agent is a small molecule that results in an increase in the lysosomal pH of the macrophage or population thereof.

37. The method of claim 34, wherein the agent is chloroquine or a derivative thereof.

38. The method of any one of claims 25-37, wherein agent is a nucleic acid that reduces the activity of one or more deoxyribonucleases in the macrophage or population thereof.

39. The method of claim 38, wherein the agent is a siRNA or shRNA that selectively binds to a mRNA transcript encoding DNase II.

40. The method of any one of claims 25-39, wherein the agent is administered intravenously, orally, or through inhalation.

41. The method of any one of claims 25-40, wherein the macrophage or population thereof are in the lung.

42. The method of any one of claims 25-41, wherein the subject is a human patient.

43. The method of claim 42, wherein the subject has, is suspected of having, or is at risk for a disease.

44. The method of claim 43 wherein the disease is a cancer.

45. The method of claim 44 wherein the cancer is selected from: colorectal cancer, lung cancer, breast cancer, pancreatic cancer, prostate cancer, bladder cancer, kidney cancer, thyroid cancer, uterine cancer, cervical cancer, ovarian cancer, testicular cancer, esophageal cancer, stomach cancer, liver cancer, brain cancer, peritoneal cancer, lymphoma, leukemia, multiple myeloma, neuroblastoma, osteosarcoma, and soft tissue sarcoma.

46. The method of any one of claims 25-45, wherein obtaining the tumor DNA comprises isolating the tumor DNA from lysosomes of the macrophage or population thereof.

47. The method of any one of claims 25-46, further comprising sequencing the tumor DNA.

48. The method of any one of claims 25-47, further comprising detecting the tumor DNA.

49. The method of any one of claims 25-48, further comprising identifying the subject as having a disease if the tumor DNA is determined to contain one or more genetic markers indicative of the disease.

50. The method of any one of claims 25-49, further comprising administering to the subject one or more treatments for the disease.

51. The method of any one of claims 25-50, further comprising collecting the macrophage or population thereof.

52. The method of claim 51, further comprising isolating the tumor DNA from the macrophage or population thereof.

53. The method of claim 52, further comprising isolating the lysosomes from the macrophage or population thereof.

54. The method of claim 53, further comprising isolating the tumor DNA from the lysosomes.

55. The method of any one of claims 25-54, further comprising attenuating macrophage adherence, thereby increasing the yield of said macrophage or population thereof.

56. The method of any one of claims 25-55, further comprising increasing efferocytosis of tumor DNA by the macrophage or population thereof.

57. The method of any one of claims 25-56, comprising detecting the tumor DNA.

58. The method of any one of claims 25-57, wherein the agent is administered by inhalation as a formulation comprising aerosolized microspheres comprising said agent.

59. A method for enhancing tumor detection in a tissue, comprising administering an agent that increases the accumulation of tumor DNA in a macrophage or population thereof in the tissue, and evaluating the accumulated tumor DNA, thereby detecting the tumor.

60. The method of claim 59, wherein the macrophage or population thereof is collected from one or more biological samples.

61. The method of claim 60, wherein the macrophage or population thereof is collected from biological sample comprising sputum.

62. The method of claim 61, wherein the macrophage or population thereof are collected by sputum induction or bronchoalveolar lavage (BAL).

63. The method of any one of claims 59-62, wherein the concentration of tumor DNA in the macrophage or population thereof is increased by up to 2-fold, up to 3-fold, up to 4-fold, up to 5-fold, up to 10-fold, up to 25-fold, up to 50-fold, up to 100-fold, up to 200-fold, up to 300-fold, up to 400-fold, up to 500-fold, up to 600-fold, up to 700-fold, up to 800-fold, up to 900-fold, or up to 1000-fold.

64. The method of any one of claims 59-63, wherein the agent results in less DNA degradation in the macrophage or population thereof.

65. The method of claim 64, wherein the amount of DNA degradation is reduced by up to 5%, up to 10%, up to 15%, up to 20%, up to 25%, up to 30%, up to 35%, up to 40%, up to 45%, up to 50%, up to 55%, up to 60%, up to 65%, up to 70%, up to 75%, up to 80%, up to 85%, up to 90%, up to 95%, up to 99%, or up to 100%, as compared to the amount of DNA degradation in the absence of the agent.

66. The method of any one of claims 59-65, wherein agent is a small molecule that reduces the activity of one or more deoxyribonucleases in the population of leukocytes.

67. The method of claim 66, wherein the agent is a DNase II inhibitor.

68. The method of claim 64, wherein the agent is a small molecule that results in an increase in the lysosomal pH of the macrophage or population thereof.

69. The method of claim 68, wherein the agent is chloroquine or a derivative thereof.

70. The method of any one of claims 59-69, wherein agent is a nucleic acid that reduces the activity of one or more deoxyribonucleases in the macrophage or population thereof.

71. The method of claim 70, wherein the agent is a siRNA or shRNA that selectively binds to a mRNA transcript encoding DNase II.

72. The method of any one of claims 59-71, wherein the agent is administered by inhalation.

73. The method of any one of claims 59-72, wherein the macrophage or population thereof is an alveolar macrophage or population thereof.

74. The method of any one of claims 59-73, wherein the subject is a human patient.

75. The method of claim 74, wherein the subject has, is suspected of having, or is at risk for a disease.

76. The method of claim 75, wherein the disease is lung cancer.

77. The method of any one of claims 59-76, wherein step of evaluating is conduct at least 1 hour after the step of administering the agent.

78. The method of any one of claims 59-77, further comprising collecting the macrophage or population thereof.

79. The method of any one of claims 59-78, further comprising isolating the tumor DNA from the macrophage or population thereof.

80. The method of claim 79, further comprising isolating the lysosomes from the macrophage or population thereof.

81. The method of claim 80, further comprising isolating the tumor DNA from the lysosomes.

82. The method of any one of claims 59-81, further comprising attenuating macrophage adherence, thereby increasing the yield of said macrophage or population thereof.

83. The method of any one of claims 59-82, further comprising increasing efferocytosis of tumor DNA by the macrophage or population thereof.

84. The method of any one of claims 59-83, comprising detecting the tumor DNA.

85. The method of any one of claims 59-84, wherein the agent is administered by inhalation as a formulation comprising aerosolized microspheres comprising said agent.

86. A pharmaceutical composition for increasing the concentration of tumor DNA, comprising:
(a) an agent capable of increasing the concentration of tumor DNA in a macrophage or population thereof; and
(b) a pharmacologically acceptable excipient.

87. A kit for increasing the concentration of phagocytosed DNA in a population of macrophages in a subject, comprising:
(a) a pharmaceutical composition comprising an agent capable of increasing the concentration of tumor DNA in the macrophage or population thereof;
(b) optionally a device for administering said composition; and
(c) instructions for administering the composition.

88. The kit of claim 87, wherein the device is a meter-dosed inhaler, a soft mist inhaler, a dry powder inhaler, or a nebulizer.

89. The kit of claim 87 or 88, wherein the kit is in the format of a liquid biopsy kit.

90. A method for detecting a tumor, comprising collecting a macrophage or population thereof which are associated with the tumor, and evaluating the tumor DNA that is accumulated by the macrophage or population thereof, thereby detecting the tumor.

91. The method of claim 90, wherein the macrophage or population thereof is collected from one or more biological samples.

92. The method of claim 91, wherein the macrophage or population thereof is collected from a biological sample comprising sputum.

93. The method of claim 92, wherein the macrophage or population thereof are collected by sputum induction or bronchoalveolar lavage (BAL).

94. The method of any one of claims 90-93, wherein the tumor is in a tissue occurring in a subject.

95. The method of claim 94, wherein the subject is a human.

96. The method of claim 94 or 95, further comprising administering to the subject an effective amount of an agent that results in an increase in the accumulation tumor DNA, prior to the collection of the macrophage or population thereof from the tissue.

97. The method of claim 96, wherein the concentration of tumor DNA in the macrophage or population thereof is increased by up to 2-fold, up to 3-fold, up to 4-fold, up to 5-fold, up to 10-fold, up to 25-fold, up to 50-fold, up to 100-fold, up to 200-fold, up to 300-fold, up to 400-fold, up to 500-fold, up to 600-fold, up to 700-fold, up to 800-fold, up to 900-fold, or up to 1000-fold.

98. The method of claim 96 or 97, wherein the agent results in less DNA degradation in the macrophage or population thereof.

99. The method of claim 98, wherein the amount of DNA degradation is reduced by up to 5%, up to 10%, up to 15%, up to 20%, up to 25%, up to 30%, up to 35%, up to 40%, up to 45%, up to 50%, up to 55%, up to 60%, up to 65%, up to 70%, up to 75%, up to 80%, up to 85%, up to 90%, up to 95%, up to 99%, or up to 100%, as compared to the amount of DNA degradation in the absence of the agent.

100. The method of any one of claims 96-99, wherein agent is a small molecule that reduces the activity of one or more deoxyribonucleases in the macrophage or population thereof.

101. The method of claim 100, wherein the agent is a DNase II inhibitor.

102. The method of any one of claims 96-99, wherein the agent is a small molecule that results in an increase in the lysosomal pH of the macrophage or population thereof.

103. The method of claim 102, wherein the agent is chloroquine or a derivative thereof.

104. The method of any one of claims 96-99, wherein agent is a nucleic acid that reduces the activity of one or more deoxyribonucleases in the macrophage or population thereof.

105. The method of claim 104, wherein the agent is a siRNA or shRNA that selectively binds to a mRNA transcript encoding DNase II.

106. The method of any one of claims 96-105, wherein the agent is administered by inhalation.

107. The method of claim 106, wherein the agent is administered via a formulation comprising aerosolized microspheres comprising said agent.

108. The method of any one of claims 96-107, wherein the macrophage or population thereof is collected at least 1 hour after the step of administering the agent.

109. The method of any one of claims 90-108, further comprising attenuating macrophage adherence prior to collection of the macrophage or population thereof, thereby increasing the yield of said macrophage or population thereof.

110. The method of any one of claims 90-109, further comprising increasing efferocytosis of tumor DNA by the macrophage or population thereof prior to collection of the macrophage or population thereof.

111. The method of any one of claims 90-110, wherein the tissue is lung tissue.

112. The method of any one of claims 90-111, wherein the macrophage or population thereof is an alveolar macrophage or population thereof.

113. The method of any one of claims 90-112, wherein the tumor is a lung cancer tumor.

114. The method of any one of claims 90-113, further comprising isolating the tumor DNA from the macrophage or population thereof, prior to evaluation of the tumor DNA.

115. The method of claim 114, further comprising isolating the lysosomes from the macrophage or population thereof.

116. The method of claim 115, further comprising isolating the tumor DNA from the lysosomes.
